(19) United States
(12) Patent Application Publication
(10) Pub. No.: US 2025/0334587 A1
(43) Pub. Date: Oct. 30, 2025

(54) USE OF EMAPALUMAB FOR TREATMENT

(71) Applicant: Swedish Orphan Biovitrum AG, Basel (CH)

(72) Inventors: Evangelos J. GIAMARELLOS-BOURBOULIS, Athens (GR); Simon Jonas ALFANO, Waltham, MA (US); Brian David JAMIESON, Waltham, MA (US); Emmanuel Pierre MONNET, Basel (CH)

(21) Appl. No.: 19/189,804

(22) Filed: Apr. 25, 2025

Related U.S. Application Data
(60) Provisional application No. 63/639,216, filed on Apr. 26, 2024.
(60) Provisional application No. 63/676,024, filed on Jul. 26, 2024.
(60) Provisional application No. 63/728,240, filed on Dec. 5, 2024.

(30) Foreign Application Priority Data
Apr. 26, 2024 (EP) 24386049.1

(51) Int. Cl. G01N 33/68; C07K 16/24

(52) U.S. Cl. CPC G01N 33/6866; C07K 16/249; G01N 2333/522; G01N 2333/57; G01N 2800/26; G01N 2800/52

(57) ABSTRACT

The instant disclosure relates to methods of treating sepsis by administering emapalumab. Also provided are diagnostic biomarkers for sepsis.

### CROSS-REFERENCE TO RELATED APPLICATIONS

The present application claims the benefit of U.S. Provisional Patent Applications No. 63/639,216, filed Apr. 26, 2024, No. 63/676,024, filed Jul. 26, 2024, and No. 63/728,240, filed Dec. 5, 2024, and EP Application Serial No. 24386049.1, filed Apr. 26, 2024, each of which is incorporated herein by reference in its entirety.

### INCORPORATION-BY-REFERENCE OF SEQUENCE LISTING

The Sequence Listing XML associated with this application is provided electronically in XML file format and is hereby incorporated by reference into the specification. The name of the XML file containing the Sequence Listing XML is “EMAC-004_001US_SeqList.xml”. The XML file is 7991 bytes, created on Apr. 14, 2025, and is being submitted electronically via USPTO Patent Center.

### FIELD

The instant disclosure relates to methods of treating sepsis by administering emapalumab. Also provided are diagnostic biomarkers for sepsis.

### BACKGROUND

Sepsis is a life-threatening organ dysfunction due to a dysregulated host response to infection (Singer et al., JAMA 2016; 315:801-10). In 2017, it was estimated that almost 50 million people suffer from sepsis worldwide, and 11 million people die from sepsis that year, representing almost 20% of all global deaths (Rudd et al., Lancet 2020; 395:200-211). Early antimicrobial treatment is key in management, but mortality remains high. The current concept is that patients with sepsis and organ dysfunctions present with large differences in their immune endotypes and that they should receive precision treatment according to this endotype (Maslove et al., Nat Med 2022; 28:141-1148).

Macrophage activation-like syndrome (MALS) is a well-characterized immune endotype of sepsis. In one post-hoc analysis of one randomized controlled trial conducted during the 90's, some of the sepsis participants were considered to have signs compatible with macrophage activation syndrome (MAS) or secondary hemophagocytic lymphohistiocytosis (sHLH) and respond better to anakinra treatment. Favorable anakinra responses led to consider that this entity was driven by the excess production of interleukin (IL)-1 by tissue macrophages (Shakoory et al., Crit Care Med 2016; 44:275-281). It was later defined that this entity should be renamed into MALS which is classified by concentrations of ferritin exceeding 4,420 ng/ml (Kyriazopoulou et al., BMC Med 2017; 15:172; Leventogiannis et al., Cell Rep Med 2022; 3: 100817). MALS is a state of hyper-inflammatory sepsis which is driven by IL-1. However, there are several more patients with rapid progression into death with levels of circulating ferritin lower than 4,420 ng/ml. It is suspected that there exists a significant proportion of patients with hyper-inflammatory sepsis which is not driven by IL-1.

Activation of the interferon-gamma (IFNγ) cascade is key in the pathophysiology of primary and secondary HLH (Karakike et al., Front Immunol 2019; 10:55) and patients with secondary HLH in the field of systemic juvenile idiopathic arthritis (sJIA) and adult-onset Still's disease (AOSD) showed significant improvement following treatment with the monoclonal antibody emapalumab, which specifically targets IFNγ (De Benedetti et al., Ann Rheum Dis 2023; 82:857-865). Several antibodies targeting IFNγ have been described but have shown varying levels of success, with some obtaining regulatory approval (e.g., emapalumab) and others not completing clinical trials.

However, the mechanisms of endotypes underlying sepsis remain poorly understood and more effective treatments are needed. Provided herein are biomarkers for a previously undefined endotype of IFNγ-driven sepsis (IDS), as well as methods of treating sepsis using emapalumab.

### SUMMARY

In one aspect, provided herein is a method of treating sepsis in a subject in need thereof, comprising administering to the subject a first dose of emapalumab, wherein the subject has a blood concentration of CXCL9 of more than 2200 pg/ml and detectable levels of IFNγ in the blood prior to the administration of the first dose of emapalumab. In some embodiments, the first dose of emapalumab is a dose of about 6 mg/kg.

In some embodiments, the method further comprises administering a second dose of emapalumab. In some embodiments, the second dose is a dose of about 3 mg/kg. In some embodiments, the second dose is a dose of about 6 mg/kg. In some embodiments, the second dose is administered three days after the first dose. In some embodiments, the method further comprises administering a third dose of emapalumab. In some embodiments, the third dose is a dose of 3 mg/kg. In some embodiments, the third dose is a dose of 6 mg/kg. In some embodiments, the third dose is administered three days after the second dose.

In some embodiments, the method further comprises administering subsequent doses of emapalumab to the subject. In some embodiments, the subsequent doses of emapalumab are administered to the subject every three days. In some embodiments, the subsequent doses of emapalumab are doses of 3 mg/kg each. In some embodiments, the emapalumab is administered for a total period of 21-28 days. In some embodiments, the subject has a HLA-DR expression of 8000 receptors/CD14-monocyte or more prior to the administration of the first dose of emapalumab.

In another aspect, provided herein is a method of identifying a subject for treatment with emapalumab, comprising: (a) obtaining a sample from the subject; (b) measuring the level of IFNγ and CXCL9 in the sample; and (c) if the levels of IFNγ is detectable and the level of CXCL9 more than 2200 pg/mL, administering at least one dose of emapalumab to the subject. In some embodiments, step (c) comprises monitoring the subject or administering a therapy not comprising emapalumab if the level of IFNγ is undetectable or the level of CXCL9 is 2200 pg/ml or less.

In some embodiments, the method further comprises repeating administration of the at least one dose of emapalumab. In some embodiments, the method further comprises repeating the administration of the at least one dose of emapalumab three times. In some embodiments, the method further comprises administering the at least one dose of emapalumab every three days for 9 days. In some embodiments, the method further comprises measuring the level of CXCL9 at regular intervals after the administration of the at least one dose of emapalumab and, if the level of CXCL9 is more than 500 pg/ml, administering a subsequent dose of emapalumab to the subject. In some embodiments, if the level of CXCL9 is 500 pg/ml or less, the subject is monitored or treated with a therapy not comprising emapalumab. In some embodiments, the level of CXCL9 is measured after the three doses of emapalumab have been administered to the subject.

In some embodiments, the method further comprises measuring the expression of HLA-DR on CD14 monocytes at regular intervals after the administration of the at least one dose of emapalumab and, if the expression of HLA-DR is 6000 receptors/CD14 monocyte or more, administering a subsequent dose of emapalumab to the subject. In some embodiments, if the expression of HLA-DR is less than 6000 receptor/monocyte, the subject is monitored or treated with a therapy not comprising emapalumab. In some embodiments, the at least one dose of emapalumab is a dose of about 8 mg/kg. In some embodiments, the at least one dose of emapalumab is a dose of about 6 mg/kg. In some embodiments, the subsequent dose of emapalumab is a dose of about 3 mg/kg. In some embodiments, the subject has an expression of HLA-DR of 8000 receptors/CD14-monocyte or more prior to the administration of the at least one dose of emapalumab. In some embodiments, the subject has specific values of an integrated score comprising blood C-reactive protein, procalcitonin, and/or ferritin prior to the administration of the at least one dose of emapalumab.

In another aspect, provided herein is a method of identifying a subject for treatment with emapalumab, comprising: (a) providing a sample obtained from the subject; (b) measuring the level of IFNγ and CXCL9 in the sample; and (c) (i) if the levels of IFNγ is detectable and the level of CXCL9 more than 2200 pg/mL, the subject is suitable for treatment by the administration of at least one dose of emapalumab; or (ii) if the level of IFNγ is undetectable or the level of CXCL9 is 2200 pg/ml or less, the subject is monitored or is suitable for treatment with a therapy not comprising emapalumab. In some embodiments, the method further comprises measuring the level of CXCL9 at regular intervals after the administration of the at least one dose of emapalumab and, if the level of CXCL9 is more than 500 pg/ml, the subject is suitable for the administration of a subsequent dose of emapalumab, or if the level of CXCL9 is 500 pg/ml or less, the subject is monitored or is suitable for treatment with a therapy not comprising emapalumab. In some embodiments, the method further comprises measuring the expression of HLA-DR at regular intervals after the administration of the at least one dose of emapalumab and, if the expression of HLA-DR is 6000 receptors/CD14 monocyte or more, the subject is suitable for the administration of a subsequent dose of emapalumab to the subject, or if the expression of HLA-DR is less than 6000 receptor/monocyte, the subject is monitored or is suitable for treatment with a therapy not comprising emapalumab. In some embodiments, the method is an in vitro method.

In another aspect, provided herein is a method of diagnosing IFNγ-driven sepsis (IDS) in a subject, comprising: (a) measuring the level of IFNγ and CXCL9 in a sample obtained from the subject; and (b) if the levels of IFNγ is detectable and the level of CXCL9 is more than 2200 pg/mL, the subject has IDS. In some embodiments, the method is an in vitro method.

### BRIEF DESCRIPTION OF DRAWINGS

FIGS. 1A-1E show the development of IFNγ-driven sepsis (IDS) endotype. FIG. 1A is a bar graphs of 28-day mortality in patients with IFNγ more than 3 pg/ml and IFNγ 3 pg/ml or less. The p-value of comparison by the Fisher's exact test is provided. FIG. 1B is a scatter plot of the absolute count of HLA-DR receptors on CD45/CD14-monocytes in patients with IFNγ more than 3 pg/ml and IFNγ 3 pg/ml or less. Lines represent medians and interquartile range. The p-value of comparison by the Mann-Whitney U test is provided. FIG. 1 C shows plots of sCD163 in patients with IFNγ more than 3 pg/ml and IFNγ 3 pg/ml or less. Lines represent medians and interquartile range. The p-value of comparison by the Mann-Whitney U test is provided. FIG. 1D is a scatter plot of CXCL9 in survivors and non-survivors. This comparison involves only the patients with IFNγ levels more than 3 pg/ml. Lines represent medians and interquartile range. The p-value of comparison by the Mann-Whitney U test is provided. CI, confidence interval; IDS, IFNγ-driven sepsis; IFN, interferon; n, number of patients; SE, standard error.

FIGS. 2A-2F show Sepsis patients classification by endotypes. FIGS. 2A-2D show shows the frequency of the co-existence of HBD and DIC, circulating concentrations of triglycerides, circulating concentrations of ferritin, and HScore (hemophagocytosis score), respectively, in patients with the MALS endotype of the discovery set. The co-presence of HBD and DIC, triglyceride concentrations, ferritin concentrations and HScores are higher in patients with MALS. Nil patient without MALS presents with both HBD and DIC. The absolute numbers of patients are shown. The p-value of comparisons by the Fisher's exact test (FIG. 2A) or the Mann-Whitney U test (FIGS. 2B-2D) is provided. Lines on top of the distribution represent medians and interquartile range. FIGS. 2E and 2F show distribution of endotypes in the discovery and the validation sets of patients, respectively. Patients are classified into the MALS and IDS endotypes. Patients with low IFNγ (i.e. ≤3 pg/ml) are presented as an independent endotype most likely representing patients with sepsis-induced immunoparalysis. The overlaps between MALS and IDS in each set are provided. In the discovery set, 126 patients with MALS have non-detectable IFNγ. IDS, IFNγ-driven sepsis; IFN, interferon; CI, confidence interval; DIC, disseminated intravascular coagulation; HBD, hepatobiliary dysfunction; MALS, macrophage activation-like syndrome; n, number of patients. CI: confidence intervals; HS: hemophagocytosis; IDS: endotype of IFNγ-driven sepsis; MALS: macrophage activation-like syndrome; n: number of patients; SE: standard error; CO: 95% confidence interval.

FIGS. 3A-3C show blood levels of C-reactive protein, blood levels of procalcitonin and plasma levels of D-dimers, respectively, in patients of the discovery set according to classification into endotypes. Lines on top of the distribution represent medians and interquartile ranges. Only statistically significant differences are shown. Comparisons are done by the Mann-Whitney U test after corrections by Bonferroni. Note the absence of difference between patients with MALS and IDS in FIGS. 3A and 3B. No statistically significant differences were found between the endotypes in FIG. 3C. IDS, IFNγ-driven sepsis; IFN, interferon; MALS, macrophage activation-like syndrome; n, number of patients; w/o, without CXCL9 increase.

FIGS. 4A and 4B show blood levels of ferritin and IL-18 in in patients of the discovery set according to classification into endotypes, respectively. Lines on top of the distributions represent medians and interquartile ranges. Only statistically significant differences are shown. Comparisons are done by the Mann-Whitney U test after corrections by Bonferroni. IDS, IFNγ-driven sepsis; IFN, interferon; MALS, macrophage activation-like syndrome; n, number of patients; w/o, without CXCL9 increase.

FIG. 5 shows blood levels of CXCL9 in patients of the discovery set according to classification into endotypes. Lines on top of the distributions represent medians and interquartile ranges. Only statistically significant differences are shown. Comparisons are done by the Mann-Whitney U test after corrections by Bonferroni. IDS, IFNγ-driven sepsis; IFN, interferon; MALS, macrophage activation-like syndrome; n, number of patients; w/o, without CXCL9 increase.

FIGS. 6A and 6B show the impact of endotype of IFNγ-driven sepsis on mortality the first 28 days. FIGS. 3A and 3B show Kaplan-Meier survival curves in the discovery and validation sets, respectively, according to sepsis endotype. The values of the indicated log-rank comparisons and of the respective p-values are provided. n: number of patients; IDS: IFNγ-driven sepsis; IFN: interferon; MALS: macrophage activation-like syndrome.

FIGS. 7A and 7B show blood levels of IFNγ in patients of the discovery set and the validation set, respectively, according to classification into endotypes. Lines on top of the distribution represent medians and interquartile ranges. Only statistically significant differences are shown. Comparisons are done by the Mann-Whitney U test after corrections by Bonferroni. IDS, IFNγ-driven sepsis; IFN, interferon; MALS, macrophage activation-like syndrome; n, number of patients; w/o, without CXCL9 increase.

FIG. 8 shows the mortality risk by the presence of the IFNγ-driven sepsis endotypes in patient sub-groups. The odds ratios of the impact of IDS on 28-day mortality coming from logistic regression analyses within each indicated subgroups is provided. The p-values of significances are shown. CAP: community-acquired pneumonia; CI: confidence interval; HAP: hospital-acquired pneumonia; HR: hazard ratio; ICU: Intensive Care Unit: VAP: ventilator-associated pneumonia.

FIGS. 9A-9C show the change of circulating concentrations of IFNγ over time in patients with the IDS sepsis endotype and 28-day mortality FIGS. 9A and 9B show paired changes of IFNγ and CXCL9, respectively between the two time points, separately for 28-day survivors and non-survivors. The p-value of paired comparisons by the Wilcoxon's test is provided. FIG. 9C shows 28-day mortality for patients with less than 50% decrease of blood IFNγ and for patients with ≥50% decrease of blood IFNγ. The 50% cut-off is defined by the Youden index following ROC curve analysis of the relative changes of IFNγ to predict 28-day mortality (area under the curve 0.69; 95% confidence intervals 0.55 to 0.82; p=0.016). CI: confidence interval; IDS: IFNγ-driven sepsis; IFN; interferon; OR: odds ratio; n: number of patients.

FIG. 10 shows the study design of the EMBRACE randomized controlled trial. Each arrow of the groups emapalumab 1 and 2 indicates one dose of 3 mg/kg. Biomarkers: CRP, IL-6, IFNγ, ferritin and CXCL9.

### DETAILED DESCRIPTION

Provided herein are methods of treating sepsis in a subject in need thereof. The patients treated in accordance with these methods may suffer from sepsis driven by the IDS endotype. Also provided herein methods of selecting a subject for the treatment with emapalumab, comprising measuring the levels of IFNγ, CXCL9 and/or HLA-DR on CD14 monocytes in a sample from the subject.

### Methods of Treatment

In one aspect, provided herein is a method of treating sepsis in a subject in need thereof, comprising administering emapalumab to the subject. Emapalumab (GAMFIANT®) is a fully human anti-IFNγ mAb that was FDA approved in 2018 for the treatment of adult and pediatric primary hemophagocytic lymphohistiocytosis (HLH).

A person of skill in the art will appreciate that the methods disclosed herein are also useful for distinguishing patients suffering from IDS from patients suffering from different forms of sepsis, e.g., sepsis driven by another mechanism, such as. MALS, to identify subjects for treatment with emapalumab, and/or to guide treatment with emapalumab.

Emapalumab is described in more detail in U.S. Pat. No. 7,700,098, which is incorporated herein by reference in its entirety. Emapalumab has a heavy chain variable (VH) region comprising the amino acid sequence EVQLLESGGGLVQPGGSLRLSCAASGFTFSSYAMSWVRQAPGKGLEWVSAIS GSGGSTYYADSVKGRFTISRDNSKNTLYLQMNSLRAEDTAVYYCAKDGSSGWYVPHW FDPWGQGTLVTVSS (SEQ ID NO: 1) and a light chain variable (VL) region comprising the amino acid sequence NFMLTQPHSVSESPGKTVTISCTRSSGSIASNYVQWYQQRPGSSPTTVIYEDNQRPSGVP DRFSGSIDSSSNSASLTISGLKTEDEADYYCQSYDGSNRWMFGGGTKLTVL (SEQ ID NO: 2). The CDRs of emapalumab are set forth in Table 1.

TABLE 1
CDRs of Emapalumab

| | CDR1 | CDR2 | CDR3 |
|---|---|---|---|
| VH | SYAMS | AISGSGGSTYYADSVKG | DGSSGWYVPHWFDP |
| | (SEQ ID NO: 3) | (SEQ ID NO: 4) | (SEQ ID NO: 5) |
| VL | TRSSGSIASNYVQ | EDNQRPS | QSYDGSNRWM |
| | (SEQ ID NO: 6) | (SEQ ID NO: 7) | (SEQ ID NO: 8) |

### Dosing and Administration

The dose and frequency of emapalumab that should be administered to a subject may depend on the severity of the subject's condition as well as other factors, such as the subject's weight or general health. Emapalumab is generally administered based on body weight.

Depending on the severity and subject's response, emapalumab may be administered more than once. For example, the subject may be administered a first, second, third, fourth, fifth and subsequent dose. In some embodiments, the second dose is administered three days after the first dose. In some embodiments, the third dose is administered three days after the second dose. In some embodiments, the fourth dose is administered three days after the third dose. In some embodiments, the fifth dose is administered three days after the fourth dose.

In some embodiments, emapalumab is administered daily (e.g., every 24 hours) for 6 days. In some embodiments, emapalumab is administered daily (e.g., every 24 hours) for 12 days. In some embodiments, emapalumab is administered daily (e.g., every 24 hours) for 15 days. In some embodiments, emapalumab is administered daily (e.g., every 24 hours) for 18 days. In some embodiments, emapalumab is administered daily (e.g., every 24 hours) for 21 days. In some embodiments, emapalumab is administered daily (e.g., every 24 hours) for 28 days.

In some embodiments, emapalumab is administered every other day (e.g., every 48 hours) for 6 days. In some embodiments, emapalumab is administered every other day (e.g., every 48 hours) for 12 days. In some embodiments, emapalumab is administered every other day (e.g., every 48 hours) for 15 days. In some embodiments, emapalumab is administered every other day (e.g., every 48 hours) for 18 days. In some embodiments, emapalumab is administered every other day (e.g., every 48 hours) for 21 days. In some embodiments, emapalumab is administered every other day (e.g., every 48 hours) for 28 days.

In some embodiments, emapalumab is administered every three days (e.g., every 72 hours) for 6 days. In some embodiments, emapalumab is administered every three days (e.g., every 72 hours) for 12 days. In some embodiments, emapalumab is administered every three days (e.g., every 72 hours) for 15 days. In some embodiments, emapalumab is administered every three days (e.g., every 72 hours) for 18 days. In some embodiments, emapalumab is administered every three days (e.g., every 72 hours) for 21 days. In some embodiments, emapalumab is administered every three days (e.g., every 72 hours) for 28 days.

In some embodiments, emapalumab is administered every four days (e.g., every 96 hours) for 6 days. In some embodiments, emapalumab is administered every four days (e.g., every 96 hours) for 12 days. In some embodiments, emapalumab is administered every four days (e.g., every 96 hours) for 15 days. In some embodiments, emapalumab is administered every four days (e.g., every 96 hours) for 18 days. In some embodiments, emapalumab is administered every four days (e.g., every 96 hours) for 21 days. In some embodiments, emapalumab is administered every four days (e.g., every 96 hours) for 28 days.

In some embodiments, the dosing frequency may be decreases after the second, third, fourth or fifth dose. For example, a patient may receive a emapalumab every three days for five doses and then every four days.

In some embodiments, the subject is administered a dose of emapalumab of about 1 mg/kg to about 10 mg/kg. In some embodiments, the subject is administered a dose of emapalumab of about 1 mg/kg to about 5 mg/kg. In some embodiments, the subject is administered a dose of emapalumab of about 5 mg/kg to about 10 mg/kg. In some embodiments emapalumab is administered at a dose of about 1 mg/kg. In some embodiments, emapalumab is administered at a dose of about 2 mg/kg. In some embodiments, emapalumab is administered at a dose of about 3 mg/kg. In some embodiments, emapalumab is administered at a dose of about 3 mg/kg. In some embodiments, emapalumab is administered at a dose of about 5 mg/kg. In some embodiments, emapalumab is administered at a dose of about 6 mg/kg. In some embodiments, emapalumab is administered at a dose of about 10 mg/kg. In some embodiments, emapalumab is administered as a one-time dose of 1 mg/kg intravenously. In some embodiments, emapalumab is administered as a one-time dose of 2 mg/kg intravenously. In some embodiments, emapalumab is administered as a one-time dose of 3 mg/kg intravenously. In some embodiments, emapalumab is administered as a one-time dose of 4 mg/kg intravenously. In some embodiments, emapalumab is administered as a one-time dose of 5 mg/kg intravenously. In some embodiments, emapalumab is administered as a one-time dose of 6 mg/kg intravenously.

In embodiments wherein the subject is administered more than one dose of emapalumab, the doses may be different, e.g., a first dose may be a dose of 6 mg/kg and a second and subsequence dose may be a dose of 6 mg/kg or 3 mg/kg or a first dose may be a dose of 10 mg/kg and a second and subsequent dose may be a dose of 6 mg/kg or 3 mg/kg. In some embodiments wherein the subject is administered more than one dose emapalumab, the doses may be the same, e.g., the first, second, and subsequent dose may be a dose of 3 mg/kg.

If more than one dose of emapalumab is administered, it may be preferable to administer a higher dose initially followed by a maintenance dose. For example, a dose of 6 mg/kg or 10 mg/kg may be administered initially (e.g., for the first 2, 3, or 4 administrations) followed by a maintenance dose of 3 mg/kg. The maintenance dose may be administered less frequently than the initial dose. For example, the initial dose may be administered every three days while the maintenance dose is administered every four days.

In some embodiments, the first and second dose are the same and the third and subsequent dose are different from the first and second dose. For example, the first and second dose of emapalumab may be 6 mg/kg and the third and subsequent dose of emapalumab may be 3 mg/kg of emapalumab, if any.

In some embodiments, the first and second dose different and the third and subsequent dose are different from the first and second dose. For example, the first dose of emapalumab may be 10 mg/kg, the second dose may be 6 mg/kg, and the third and subsequence dose of emapalumab may be 3 mg/kg.

In some embodiments, a subject is administered a first a dose of 6 mg/kg emapalumab and a second dose of 3 mg/kg emapalumab three days after the first dose. In some embodiments, the subject is administered a third and subsequent dose of 3 mg/kg emapalumab every 3 days thereafter.

In some embodiments, a subject is administered a first a dose of 6 mg/kg emapalumab, a second dose of 6 mg/kg emapalumab three days after the first dose, a third dose of 6 mg/kg emapalumab three days after the second dose and a fourth dose of 3 mg/kg emapalumab three days after the third dose. In some embodiments, the subject is administered a fifth and subsequent dose of 3 mg/kg emapalumab every 3 days thereafter.

In some embodiments, a subject is administered a first a dose of 6 mg/kg emapalumab, a second dose of 6 mg/kg emapalumab three days after the first dose, a third dose of 6 mg/kg emapalumab three days after the second dose and then subsequence doses of 3 mg/kg emapalumab every three or four days.

Methods of treatment described herein may further comprise measuring the expression of HLA-DR on CD14 monocytes, the levels of IFNγ and/or the levels of CXCL9 in a subject to determine whether treatment should be initiated, as well as to determine whether treatment should be continued once started. HLA-DR on CD14 monocytes in this context may be used to determine if a subject is suffering from immunoparalysis. CXCL9 and IFNγ in this context may be used to determine whether the IFNγ-pathway driving the sepsis is still active. The concentrations of HLA-DR, IFNγ and CXCL9 may be determined using any suitable in vitro method known in the art or described herein.

In some embodiments, IFNγ levels that are detectable (e.g., IFNγ levels of more than about 0.78 pg/mL. more than about 1 pg/mL, more than about 2 pg/mL, more than about 3 pg/mL, more than about 4 pg/ml, more than about 5 pg/mL, more than about 10 pg/mL, more than about 15 pg/mL, more than about 20 pg/mL, or more than about 25 pg/mL) indicate that the IFNγ-pathway driving the sepsis is still active. In some embodiments, CXCL9 levels of more than about 1000 pg/ml, more than about 1200 pg/ml, more than about 1400 pg/ml, more than about 1600 pg/ml, more than about 1800 pg/ml, more than about 2000 pg/ml, more than about 2200 pg/ml, more than about 2400 pg/ml, more than about 2600 pg/ml, more than about 2800 pg/ml, more than about 3000 pg/ml, more than about 3200 pg/ml, more than about 3400 pg/ml, more than about 3600 pg/ml, more than about 3800 pg/ml or more than about 4000 pg/mL indicate that the pathway driving the sepsis is still active.

Blood concentrations of IFNγ, and CXCL9 may be measured in a blood sample from a subject using any suitable method known in the art or described herein, for example, an enzyme-linked immunosorbent assay (ELISA). Concentrations of IFNγ, and CXCL9 may be determined in any suitable sample from a subject, for example, a whole blood sample, a plasma sample or a serum sample.

In some embodiments, a method of treating sepsis disclosed herein comprises measuring the expression of HLA-DR on CD14 monocytes in a subject and, if the expression of HLA-DR is 8000 receptors/CD14-monocyte or more, administering an initial dose of emapalumab. If the expression of HLA-DR is less than 8000 receptors/CD14-monocyte, the subject may be monitored instead of receiving a subsequent dose of emapalumab, or the subject may be a treatment for sepsis not comprising emapalumab. Monitoring a subject may comprise clinical observation, further measurements of HLA-DR, or conducting other tests to determine whether emapalumab administration is indicated. HLA-DR on CD14 monocytes may be determined in any suitable sample from a subject, for example, a whole blood sample, a plasma sample or a serum sample.

In some embodiments, a method of treating sepsis disclosed herein comprises repeatedly measuring the expression of HLA-DR on CD14 monocytes in a subject receiving emapalumab. For example, levels of HLA-DR on CD14 monocytes may be measured every three days after the subject first receives emapalumab. If in one of these regular measurements the expression of HLA-DR is 6000 receptors/CD14-monocyte or more, the subject may be administered a subsequent dose of emapalumab. If in one of these regular measurements the expression of HLA-DR is less than 6000 receptors/CD14-monocyte, the subject may be monitored instead of receiving a subsequent dose of emapalumab, or the subject may be administered an alternative treatment for sepsis. HLA-DR on CD14 monocytes may be determined in any suitable sample from a subject, for example, a whole blood sample, a plasma sample or a serum sample.

In another aspect, provided herein is a method of treating sepsis in a subject comprising the steps of (a) obtaining a sample from the subject, (b) measuring the level of CXCL9 in the sample, and (c) if the CXCL9 is more than 2200 pg/ml, administering an initial dose of emapalumab to the subject. If the CXCL9 level measured in step (c) is 2200 pg/ml or less, the subject may be monitored instead of receiving emapalumab. Monitoring a subject may comprise clinical observation, further measurements of CXCL9, or conducting other tests to determine whether emapalumab administration is indicated. Alternatively, if the CXCL9 level measured in step (c) is less 2200 pg/ml or less, the subject may receive a treatment for sepsis, that does not comprise emapalumab. CXCL9 may be determined in any suitable sample from a subject, for example, a whole blood sample, a plasma sample or a serum sample.

In some embodiments, a method of treating sepsis disclosed herein comprises repeatedly measuring the level of CXCL9 in a subject receiving emapalumab. For example, levels of CXCL9 may be measured every three days after the subject first receives emapalumab. If in one of these regular measurements CXCL9 is more than 500 pg/ml, the subject may be administered a subsequent dose of emapalumab. If in one of these regular measurements the level of CXCL9 is 500 pg/ml or less, the subject may be monitored instead of receiving a subsequent dose of emapalumab, or the subject may be administered a treatment for sepsis not comprising emapalumab. CXCL9 may be determined in any suitable sample from a subject, for example, a whole blood sample, a plasma sample or a serum sample.

In another aspect, provided herein is a method of treating sepsis in a subject comprising the steps of (a) obtaining a sample from the subject, (b) measuring the level of IFNγ in the sample, and (c) if the IFNγ is detectable, administering an initial dose of emapalumab to the subject. If the IFNγ level measured in step (c) is undetectable, the subject may be monitored instead of receiving emapalumab. Monitoring a subject may comprise clinical observation, further measurements of IFNγ, or conducting other tests to determine whether emapalumab administration is indicated. Alternatively, if the IFNγ level measured in step (c) is undetectable, the subject may receive a treatment for sepsis not comprising emapalumab. IFNγ may be determined in any suitable sample from a subject, for example, a whole blood sample, a plasma sample or a serum sample. In some embodiments, an “undetectable” level of IFNγ is a level of 25 pg/mL or less.

In some embodiments, a method of treating sepsis disclosed herein comprises repeatedly measuring the level of IFNγ in a subject receiving emapalumab. For example, levels of IFNγ may be measured every three days after the subject first receives emapalumab. If in one of these regular measurements the level of IFNγ is detectable, the subject may be administered a subsequent dose of emapalumab. If in one of these regular measurements the level of IFNγ is undetectable, the subject may be monitored instead of receiving a subsequent dose of emapalumab, or the subject may be administered a treatment for sepsis not comprising emapalumab. IFNγ levels may be determined in any suitable sample from a subject, for example, a whole blood sample, a plasma sample or a serum sample. In some embodiments, an “undetectable” level of IFNγ is a level of 25 pg/mL or less.

In one aspect, provided herein is a method of treating sepsis in a subject, comprising the steps of (a) obtaining a sample from the subject, (b) measuring the level of IFNγ and the level of CXCL9 in the sample, and (c) if the level of IFNγ is detectable and the level of CXCL9 is more than 2200 pg/ml, administering an initial dose of emapalumab to the subject. If the IFNγ level measured in step (c) is undetectable or the CXCL9 level measured in step (c) is 2200 pg/ml or less, the subject may be monitored instead of receiving emapalumab. Monitoring a subject may comprise clinical observation, further measurements of IFNγ and CXCL9, or conducting other tests to determine whether emapalumab administration is indicated. Alternatively, if the IFNγ level measured in step (c) is undetectable or the CXCL9 level measured in step (c) is 2200 pg/ml or less, the subject may receive a treatment for sepsis not comprising emapalumab. CXCL9 and IFNγ may be determined in any suitable sample from a subject, for example, a whole blood sample, a plasma sample or a serum sample. In some embodiments, an “undetectable” level of IFNγ is a level of 25 pg/mL or less.

In some embodiments, a method of treating sepsis disclosed herein comprises repeatedly measuring the level of IFNγ and CXCL9 in a subject receiving emapalumab. For example, levels of IFNγ and CXCL9 may be measured every three days after the subject first receives emapalumab. If in one of these regular measurements the level of IFNγ is detectable and the level of CXCL9 is more than 500 pg/ml, the subject may be administered a subsequent dose of emapalumab. If in one of these regular measurements the level of IFNγ is undetectable or the level of CXCL9 is 500 pg/mL or less, the subject may be monitored instead of receiving a subsequent dose of emapalumab, or the subject may be administered a treatment for sepsis not comprising emapalumab. IFNγ levels may be determined in any suitable sample from a subject, for example, a whole blood sample, a plasma sample or a serum sample. In some embodiments, an “undetectable” level of IFNγ is a level of 25 pg/mL or less.

In one aspect, provided herein is a method of treating sepsis in a subject, comprising the steps of (a) obtaining a sample from the subject, (b) measuring the expression of HLA-DR on CD14 monocytes and the level of CXCL9 in the sample, and (c) if the expression of HLA-DR is 8000 receptors/CD14-monocyte and more and the level of CXCL9 is more than 2200 pg/ml, administering an initial dose of emapalumab to the subject. If the HLA-DR level measured in step (c) is less than 8000 receptors/CD14-monocyte or the CXCL9 level measured in step (c) is 2200 pg/ml or less, the subject may be monitored instead of receiving emapalumab. Monitoring a subject may comprise clinical observation, further measurements of HLA-DR on CD14 monocytes and CXCL9, or conducting other tests to determine whether emapalumab administration is indicated. Alternatively, if the HLA-DR level measured in step (c) is less than 8000 receptors/CD14-monocyte or the CXCL9 level measured in step (c) is 2200 pg/ml or less, the subject may receive a treatment for sepsis not comprising emapalumab. CXCL9 and HLA-DR on CD14 monocytes may be determined in any suitable sample from a subject, for example, a whole blood sample, a plasma sample or a serum sample.

In some embodiments, a method of treating sepsis disclosed herein comprises repeatedly measuring the levels of HLA-DR on CD14 monocytes and CXCL9 in a subject receiving emapalumab. For example, levels of HLA-DR on CD14 monocytes and CXCL9 may be measured every three days after the subject first receives emapalumab. If in one of these regular measurements the expression of HLA-DR is 6000 receptors/CD14-monocyte or more, and if the level of CXCL9 is more than 500 pg/ml, the subject may be administered a subsequent dose of emapalumab. If in one of these regular measurements the expression of HLA-DR is less than 6000 receptors/CD14-monocyte, or if the level of CXCL9 is 500 pg/ml or less, the subject may be monitored instead of receiving a subsequent dose of emapalumab, or the subject may be administered a treatment for sepsis not comprising emapalumab. CXCL9 and HLA-DR on CD14 monocytes may be determined in any suitable sample from a subject, for example, a whole blood sample, a plasma sample or a serum sample.

In one aspect, provided herein is a method of treating sepsis in a subject, comprising the steps of (a) obtaining a sample from the subject, (b) measuring the expression of HLA-DR on CD14 monocytes and the level of IFNγ in the sample, and (c) if the expression of HLA-DR is 8000 receptors/CD14-monocyte or more and the level of IFNγ is detectable, administering an initial dose of emapalumab to the subject. If the HLA-DR level measured in step (c) is less than 8000 receptors/CD14-monocyte or the IFNγ level measured in step (c) is undetectable, the subject may be monitored instead of receiving emapalumab. Monitoring a subject may comprise clinical observation, further measurements of HLA-DR on CD14 monocytes and IFNγ, or conducting other tests to determine whether emapalumab administration is indicated. Alternatively, if the HLA-DR level measured in step (c) is less than 8000 receptors/CD14-monocyte or the IFNγ level measured in step (c) is undetectable, the subject may receive a treatment for sepsis not comprising emapalumab. IFNγ and HLA-DR on CD14 monocytes may be determined in any suitable sample from a subject, for example, a whole blood sample, a plasma sample or a serum sample. In some embodiments, an “undetectable” level of IFNγ is a level of 25 pg/mL or less.

In some embodiments, a method of treating sepsis disclosed herein comprises repeatedly measuring the levels of HLA-DR on CD14 monocytes and IFNγ in a subject receiving emapalumab. For example, levels of HLA-DR on CD14 monocytes and IFNγ may be measured every three days after the subject first receives emapalumab. If in one of these regular measurements the expression of HLA-DR is 6000 receptors/CD14-monocyte or more, and if the level of IFNγ is detectable, the subject may be administered a subsequent dose of emapalumab. If in one of these regular measurements the expression of HLA-DR is less than 6000 receptors/CD14-monocyte, or if the level of IFNγ is undetectable, the subject may be monitored instead of receiving a subsequent dose of emapalumab, or the subject may be administered a treatment for sepsis not comprising emapalumab. IFNγ and HLA-DR on CD14 monocytes may be determined in any suitable sample from a subject, for example, a whole blood sample, a plasma sample or a serum sample.

In one aspect, provided herein is a method of treating sepsis in a subject, comprising the steps of (a) obtaining a sample from the subject, (b) measuring the expression of HLA-DR, CXCL9, and IFNγ in the sample, and (c) if the expression of HLA-DR is 8000 receptors/CD14-monocyte or more, the level of CXCL9 is more than 2200 pg/ml, and the level of IFNγ is detectable, administering an initial dose of emapalumab to the subject. If the HLA-DR level measured in step (c) is less than 8000 receptors/CD14-monocyte, the CXCL9 level measured in step (c) is 2200 pg/ml or less, or the IFNγ level measured in step (c) is undetectable, the subject may be monitored instead of receiving emapalumab. Monitoring a subject may comprise clinical observation, further measurements of HLA-DR, CXCL9 and IFNγ, or conducting other tests to determine whether emapalumab administration is indicated. Alternatively, if the HLA-DR level measured in step (c) is less than 8000 receptors/CD14-monocyte, the CXCL9 level measured in step (c) is 2200 pg/ml or less, or the IFNγ level measured in step (c) is undetectable, the subject may receive a treatment for sepsis not comprising emapalumab. CXCL9, HLA-DR, and IFNγ may be determined in any suitable sample from a subject, for example, a whole blood sample, a plasma sample or a serum sample.

In some embodiments, a method of treating sepsis disclosed herein comprises repeatedly measuring the levels of HLA-DR, CXCL9 and IFNγ in a subject receiving emapalumab. For example, levels of HLA-DR, CXCL9 and IFNγ may be measured every three days after the subject first receives emapalumab. If in one of these regular measurements the expression of HLA-DR is 6000 receptors/CD14-monocyte or more, the level of CXCL9 is more than 500 pg/mL, and the level of IFNγ is detectable, the subject may be administered a subsequent dose of emapalumab. If in one of these regular measurements the expression of HLA-DR is less than 6000 receptors/CD14-monocyte, the level of CXCL9 is 500 pg/mL or less, pr the level of IFNγ is undetectable, the subject may be monitored instead of receiving a subsequent dose of emapalumab, or the subject may be administered a treatment for sepsis not comprising emapalumab. IFNγ and HLA-DR on CD14 monocytes may be determined in any suitable sample from a subject, for example, a whole blood sample, a plasma sample or a serum sample.

In one aspect, provided herein is a method of treating sepsis in a subject, comprising the steps of (a) obtaining a sample from the subject, (b) measuring the expression of HLA-DR on CD14 monocytes and the level of CXCL9 in the sample, (c) if the expression of HLA-DR is less than 8000 receptors/CD14-monocyte, and if the level of CXCL9 is more than 2200 pg/ml, repeating the measuring of HLA-DR on CD14 monocytes and CXCL9 approximately 24 hours after the first measurement; and (d) administering an initial dose of emapalumab to the subject if the expression of HLA-DR is 8000 receptors/CD14-monocyte or more and if the level of CXCL9 is more than 2200 pg/mL in the repeated measurement approximately 24 hours after the first measurement. If the HLA-DR is less than 8000 receptors/CD14-monocyte and if the level of CXCL9 is more than 2200 pg/mL in the repeated measurement approximately 24 hours after the first measurement, the measuring of HLA-DR on CD14 monocytes and CXCL9 may be repeated approximately 48 hours after the first measurement. If the expression of HLA-DR is 8000 receptors/CD14-monocyte or more and if the level of CXCL9 is more than 2200 pg/ml in the repeated measurement approximately 24 hours after the first measurement, the subject may be administered an initial dose of emapalumab. If the expression of HLA-DR less than 8000 receptors/CD14-monocyte and if the level of CXCL9 is more than 2200 pg/ml in the repeated measurement approximately 48 hours after the first measurement, the subject may be monitored instead of receiving emapalumab. Monitoring a subject may comprise clinical observation, further measurements of HLA-DR on CD14 monocytes and CXCL9, or conducting other tests to determine whether emapalumab administration is indicated. Alternatively, the expression of HLA-DR less than 8000 receptors/CD14-monocyte and if the level of CXCL9 is more than 2200 pg/ml in the repeated measurement approximately 48 hours after the first measurement, the subject may receive a treatment for sepsis not comprising emapalumab. CXCL9 and HLA-DR on CD14 monocytes may be determined in any suitable sample from a subject, for example, a whole blood sample, a plasma sample or a serum sample.

A “treatment for sepsis not comprising emapalumab” in the context of the methods described herein may be any suitable treatment for sepsis. A person of skill in the art will appreciate that the suitable treatment will depend on several factors, and will be able to select a suitable treatment based on local protocols and standard of care options.

### Endpoints of Efficacy

In some aspects, a method of treatment described herein results in an improvement of a subject's sequential organ failure assessment (SOFA) score. This score assesses the likelihood of death in critically ill subjects (e.g., subjects with sepsis) based on respiratory function (as measured by ratio of arterial oxygen partial pressure to fractional inspired oxygen), coagulation (as measured by platelets), liver function (as measured by bilirubin), cardiovascular function (measured by mean arterial pressure or need of vasopressors), central nervous system function (measured by Glasgow Coma Score) and renal function (measured by creatinine levels or urine output). See Vincent et al., Intensive Care Med. 1996; 22:707-10. The SOFA score ranges from 0 (normal) to 4 (most severe) per organ function and from 0 to 24 for the patient.

In some embodiments, a method of treatment described herein improves a subject's SOFA score by one point. In some embodiments, a method of treatment described herein improves a subject's SOFA score by two points. In some embodiments, a method of treatment described herein improves a subject's SOFA score by three points. In some embodiments, a method of treatment described herein improves a subject's SOFA score by four points. In some embodiments, a method of treatment described herein results in at least 10% decrease of total SOFA score. In some embodiments, a method of treatment described herein results in at least 25% decrease of total SOFA score. In some embodiments, a method of treatment described herein results in at least 50% decrease of total SOFA score. In some embodiments, a method of treatment described herein results in at least 75% decrease of total SOFA score.

In some embodiments, a method of treatment described herein improves a subject's SOFA score by one point within 1, 2, 3, 4, 5, 6, or 7 days of the first administration of emapalumab. In some embodiments, a method of treatment described herein improves a subject's SOFA score by two points within 1, 2, 3, 4, 5, 6, or 7 days of the first administration of emapalumab. In some embodiments, a method of treatment described herein improves a subject's SOFA score by three points within 1, 2, 3, 4, 5, 6, or 7 days of the first administration of emapalumab. In some embodiments, a method of treatment described herein improves a subject's SOFA score by four points within 1, 2, 3, 4, 5, 6, or 7 days of the first administration of emapalumab. In some embodiments, a method of treatment described herein results in a SOFA score of 0 within 1, 2, 3, 4, 5, 6, or 7 days of the first administration of emapalumab.

In some embodiments, a method of treatment described herein improves a subject's SOFA score by 10% within 1, 2, 3 or 4 weeks of the first administration of emapalumab. In some embodiments, a method of treatment described herein improves a subject's SOFA score by 25% within 1, 2, 3 or 4 weeks of the first administration of emapalumab. In some embodiments, a method of treatment described herein improves a subject's SOFA score by three points within 1, 2, 3 or 4 weeks of the first administration of emapalumab. In some embodiments, a method of treatment described herein improves a subject's SOFA score by four points within 1, 2, 3 or 4 weeks of the first administration of emapalumab. In some embodiments, a method of treatment described herein results in a SOFA score of 0 within 1, 2, 3 or 4 weeks of the first administration of emapalumab.

In some embodiments, a method of treatment provided herein results in survival of at least 7, 14, 21 or 28 days from the onset of sepsis. In some embodiments, a method of treatment provided herein results in survival of at least 3, 4, 5, 6, 7, or 8 weeks from the onset of sepsis.

### Indications and Subjects

In some aspects, the methods of treatment disclosed herein may be used to treat sepsis. In some embodiments, the sepsis is interferon gamma (IFNγ)-driven sepsis. It is hypothesized that IFNγ-driven sepsis is a distinct endotype of sepsis that is determined based on blood concentrations of IFNγ and CXCL9. Blood concentrations of IFNγ, and CXCL9 may be measured in a blood sample from a subject using any suitable method known in the art or described herein, for example, an enzyme-linked immunosorbent assay (ELISA). Concentrations of IFNγ, and CXCL9 may be determined in any suitable sample from a subject, for example, a whole blood sample, a plasma sample or a serum sample. IFNγ-driven sepsis is believed to distinct from MALS which is characterized by increases in markers other than CXCL9 and IFNγ, such as IL-1 and ferritin. IFNγ and CXCL9 may be used to identify subjects suffering from IFNγ-driven sepsis for treatment with emapalumab, as well as to determine whether to stop or continue treatment with emapalumab, once started. Thus, if blood concentrations of IFNγ and CXCL9 in a subject treated with emapalumab are above the thresholds described herein, the subject may require a subsequent dose of emapalumab. The subject dose may be lower, higher or the same as the initial dose. If IFNγ and CXCL9 are no longer above the threshold levels for described herein, treatment with emapalumab may be stopped.

In certain embodiments, a subject treated in accordance with the methods described herein has detectable levels of IFNγ in the blood. “Detectable” can refer to detection by any standard assay known in the art (such as an ELISA). A person of skill will appreciate that the lower limit of detection varies between assays. In certain embodiments, a subject treated in accordance with a method described herein has a blood concentration of IFNγ of more than about 1 pg/mL, more than about 2 pg/mL, more than about 3 pg/mL, more than about 4 pg/ml, more than about 5 pg/mL, more than about 10 pg/mL, more than about 15 pg/mL, more than about 20 pg/mL, or more than about 25 pg/mL prior to the administration of the first dose of emapalumab. In certain embodiments, a subject treated in accordance with a method described herein has a blood concentration of IFNγ of more than about 25 pg/ml prior to the administration of the first dose of emapalumab.

In certain embodiments, a subject treated in accordance with a method described herein has a blood concentration of CXCL9 of more than about 1000 pg/ml, more than about 1200 pg/ml, more than about 1400 pg/ml, more than about 1600 pg/ml, more than about 1800 pg/ml, more than about 2000 pg/ml, more than about 2200 pg/ml, more than about 2400 pg/ml, more than about 2600 pg/ml, more than about 2800 pg/ml, more than about 3000 pg/ml, more than about 3200 pg/ml, more than about 3400 pg/ml, more than about 3600 pg/ml, more than about 3800 pg/ml or more than about 4000 pg/mL prior to the administration of the first dose of emapalumab. In certain embodiments, a subject treated in accordance with a method described herein the subject has a blood concentration of CXCL9 of more than about 2200 pg/ml prior to the administration of the first dose of emapalumab.

In some embodiments, a subject treated in accordance with the methods described herein does not have sepsis-induced immunoparalysis. Sepsis-induced immunoparalysis may be defined as less than 8000 receptors of HLA-DR on CD14-monocytes. In some embodiments, a subject treated in accordance with a method described herein has expression of HLA-DR of at least about 8000/CD14 monocyte prior to the administration of the first dose of emapalumab. In some embodiments, a subject treated in accordance with a method described herein has a blood expression of HLA-DR of at least about 6000/CD14 monocyte prior to the administration of a subsequent dose of emapalumab.

In some embodiments, a subject treated in accordance with the methods described herein has sepsis due to infection of the lung (e.g., community-acquired pneumonia, health-care associate pneumonia, hospital-acquired pneumonia, or ventilator-associated pneumonia). In some embodiments, a subject treated in accordance with the methods described herein has sepsis due to intrabdominal infection, acute pyelonephritis or primary bacteremia or any other type. Diagnosis of infections may be done by generally accepted clinical and/or microbiological criteria.

In some embodiments, a subject treated in accordance with the methods described herein is diagnosed with or suspected of having a viral infection. In some embodiments, a subject treated in accordance with the methods described herein is diagnosed with or suspected of having a SARS-COV-2 infection. In some embodiments, a subject treated in accordance with the methods described herein is diagnosed with or suspected of having an influenza infection (e.g., an influenza A infection). In some embodiments, a subject treated in accordance with the methods described herein is diagnosed with or suspected of having an acute infection (e.g., a an infection with a not yet defined viral strain). In some embodiments, a subject treated in accordance with the methods described herein is diagnosed with or suspected of having an acute viral infection.

In some embodiments, a subject treated in accordance with the methods described herein does not have an infection with the human immunodeficiency virus. In some embodiments, a subject treated in accordance with the methods described herein does not have an end-stage solid or hematologic malignancy. In some embodiments, a subject treated in accordance with the methods described herein does not have neutropenia. In some embodiments, a subject treated in accordance with the methods described herein has not undergone organ or tissue transplantation (e.g. liver, kidney or stem cells).

The terms “patient” and “subject” are used interchangeably herein. In some embodiments, the subject is a human adult. In some embodiments, the subject is a human child.

### Biomarkers

Also provided here are methods of identifying subjects for the treatment with emapalumab. In some embodiments, the subject has sepsis (e.g., IFNγ-driven sepsis diagnosed by blood concentrations of IFNγ and/or CXCL9 as described above). Biomarkers include blood concentrations of IFNγ, CXCL9 and/or HLA-DR. As described above, HLA-DR on CD14 monocytes may be used to determine whether a subject is suffering from immunoparalysis, and CXCL9 and IFNγ may be used to determine whether the IFNγ pathways driving the sepsis is still active. Other biomarkers or assays may also be useful in determining whether a subject suffers from IFNγ-driven sepsis, as outlined below. In some embodiments, the methods of identifying subjects for the treatment with emapalumab is performed in vitro.

CXCL9, IFNγ and HLA-DR on CD14 monocytes may be determined in any suitable sample from a subject, for example, a whole blood sample, a plasma sample or a serum sample. In some embodiments of the methods disclosed herein, the measurements are performed in vitro.

In one aspect, a method of identifying a subject for treatment with emapalumab comprises the steps of (a) obtaining a sample from the subject and (b) measuring the expression of HLA-DR on CD14 monocytes in the sample. If the HLA-DR expression is 8000 receptors/CD14-monocyte or more provided, the subject may be determined suitable for emapalumab treatment and an initial dose of emapalumab may be administered to the subject. If the HLA-DR expression is less than 8000 receptors/CD14-monocyte, the subject may be determined unsuitable for emapalumab treatment. A subject deemed unsuitable for emapalumab treatment may be monitored instead of receiving emapalumab. Monitoring a subject may comprise clinical observation, further measurements of HLA-DR, or conducting other tests to determine whether emapalumab administration is indicated. Alternatively, if the HLA-DR expression is less than 8000 receptors/CD14-monocyte the subject may be deemed unsuitable for emapalumab treatment and the subject may receive a treatment for sepsis not comprising emapalumab.

In one aspect, a method of identifying a subject for continued treatment with emapalumab comprises repeatedly measuring the expression of HLA-DR on CD14 monocytes in a patient receiving emapalumab. For example, levels of HLA-DR on CD14 monocytes may be measured every three days after the subject first receives emapalumab. If in one of these regular measurements the expression of HLA-DR is 6000 receptors/CD14-monocyte or more, the subject may be deemed suitable for continued treatment with emapalumab and the subject may be administered a subsequent dose of emapalumab. If in one of these regular measurements the expression of HLA-DR is less than 6000 receptors/CD14-monocyte, the subject may be deemed unsuitable for continued emapalumab treatment and may be monitored or receive a treatment for sepsis not comprising emapalumab.

In one aspect, a method of identifying a subject for treatment with emapalumab comprises the steps of (a) obtaining a sample from the subject and (b) measuring the level of CXCL9 in the sample. If the CXCL9 level is more than 2200 pg/ml, the subject may be determined suitable for emapalumab treatment and an initial dose of emapalumab may be administered to the subject. If the CXCL9 level is 2200 pg/mL or less, the subject may be determined unsuitable for emapalumab treatment. A subject deemed unsuitable for emapalumab treatment may be monitored instead of receiving emapalumab. Monitoring a subject may comprise clinical observation, further measurements of CXCL9, or conducting other tests to determine whether emapalumab administration is indicated. Alternatively, if the CXCL9 level is 2200 pg/mL or less, the subject may be deemed unsuitable for emapalumab treatment and the subject may receive a treatment for sepsis not comprising emapalumab.

In one aspect, a method of identifying a subject for continued treatment with emapalumab comprises repeatedly measuring the level of CXCL9 in a patient receiving emapalumab. For example, levels of CXCL9 may be measured every three days after the subject first receives emapalumab. If in one of these regular measurements the level of CXCL9 is more than 500 pg/mL, the subject may be deemed suitable for continued treatment with emapalumab and the subject may be administered a subsequent dose of emapalumab. If in one of these regular measurements the level of CXCL9 is 500 pg/mL or less, the subject may be deemed unsuitable for continued emapalumab treatment and may be monitored or receive a treatment for sepsis not comprising emapalumab.

In one aspect, a method of identifying a subject for treatment with emapalumab comprises the steps of (a) obtaining a sample from the subject and (b) measuring the level of IFNγ in the sample. If the IFNγ level is detectable, the subject may be determined suitable for emapalumab treatment and an initial dose of emapalumab may be administered to the subject. If the IFNγ level is undetectable, the subject may be determined unsuitable for emapalumab treatment. A subject deemed unsuitable for emapalumab treatment may be monitored instead of receiving emapalumab. Monitoring a subject may comprise clinical observation, further measurements of IFNγ, or conducting other tests to determine whether emapalumab administration is indicated. Alternatively, if the IFNγ level is undetectable, the subject may be deemed unsuitable for emapalumab treatment and the subject may receive a treatment for sepsis not comprising emapalumab. In some embodiments, an “undetectable” level of IFNγ is a level of 25 pg/mL or less.

In one aspect, a method of identifying a subject for continued treatment with emapalumab comprises repeatedly measuring the level of IFNγ in a patient receiving emapalumab. For example, levels of IFNγ may be measured every three days after the subject first receives emapalumab. If in one of these regular measurements the level of IFNγ is detectable, the subject may be deemed suitable for continued treatment with emapalumab and the subject may be administered a subsequent dose of emapalumab. If in one of these regular measurements the level of IFNγ is undetectable, the subject may be deemed unsuitable for continued emapalumab treatment and may be monitored or receive a treatment for sepsis not comprising emapalumab. In some embodiments, an “undetectable” level of IFNγ is a level of 25 pg/mL or less.

In one aspect, a method of identifying a subject for treatment with emapalumab comprises the steps of (a) obtaining a sample from the subject and (b) measuring the expression of HLA-DR on CD14 monocytes and the level of CXCL9 in the sample. If the expression of HLA-DR on CD14 monocytes is 8000 receptors/CD14-monocyte or more and the level of CXCL9 is more than 2200 pg/ml, the subject may be determined suitable for emapalumab treatment and an initial dose of emapalumab may be administered to the subject. If the expression of HLA-DR on CD14 monocytes is less than 8000 receptors/CD14-monocyte or the level of CXCL9 is 2200 pg/ml or less, the subject may be determined unsuitable for emapalumab treatment. A subject deemed unsuitable for emapalumab treatment may be monitored instead of receiving emapalumab. Monitoring a subject may comprise clinical observation, further measurements of CXCL9 and HLA-DR, or conducting other tests to determine whether emapalumab administration is indicated. Alternatively, if the expression of HLA-DR on CD14 monocytes is less than 8000 receptors/CD14-monocyte or the level of CXCL9 is 2200 pg/ml or less, the subject may be deemed unsuitable for emapalumab treatment and the subject may receive a treatment for sepsis not comprising emapalumab.

In one aspect, a method of identifying a subject for continued treatment with emapalumab comprises repeatedly measuring the expression of HLA-DR on CD14 monocytes and the level of CXCL9 in a patient receiving emapalumab. For example, levels of HLA-DR on CD14 monocytes and CXCL9 may be measured every three days after the subject first receives emapalumab. If in one of these regular measurements the expression of HLA-DR on CD14 monocytes is 6000 receptors/CD14-monocyte or more and the level of CXCL9 is more than 500 pg/ml, the subject may be deemed suitable for continued treatment with emapalumab and the subject may be administered a subsequent dose of emapalumab. If in one of these regular measurements the expression of HLA-DR on CD14 monocytes is less than 6000 receptors/CD14-monocyte or the level of CXCL9 is 500 pg/ml or less, the subject may be deemed unsuitable for continued emapalumab treatment and may be monitored or receive a treatment for sepsis not comprising emapalumab.

In one aspect, a method of identifying a subject for treatment with emapalumab comprises the steps of (a) obtaining a sample from the subject and (b) measuring the level of IFNγ and the level of CXCL9 in the sample. If the level of IFNγ is detectable and if the level of CXCL9 is more than 2200 pg/ml, the subject may be determined suitable for emapalumab treatment and an initial dose of emapalumab may be administered to the subject. If the IFNγ level is undetectable or the CXCL9 level is 2200 pg/ml or less, the subject may be determined unsuitable for emapalumab treatment. A subject deemed unsuitable for emapalumab treatment may be monitored instead of receiving emapalumab. Monitoring a subject may comprise clinical observation, further measurements of CXCL9 and IFNγ, or conducting other tests to determine whether emapalumab administration is indicated. Alternatively, if the IFNγ level is undetectable or the CXCL9 level is 2200 pg/ml or less, the subject may be deemed unsuitable for emapalumab treatment and the subject may receive a treatment for sepsis not comprising emapalumab. In some embodiments, an “undetectable” level of IFNγ is a level of 25 pg/mL or less.

In one aspect, a method of identifying a subject for continued treatment with emapalumab comprises repeatedly measuring the level of IFNγ and the level of CXCL9 in a patient receiving emapalumab. For example, levels of IFNγ and CXCL9 may be measured every three days after the subject first receives emapalumab. If in one of these regular measurements if the level of IFNγ is detectable and if the level of CXCL9 is more than 2200 pg/ml, the subject may be deemed suitable for continued treatment with emapalumab and the subject may be administered a subsequent dose of emapalumab. If in one of these regular measurements the IFNγ level is undetectable or the CXCL9 level is 2200 pg/ml or less, the subject may be deemed unsuitable for continued emapalumab treatment and may be monitored or receive a treatment for sepsis not comprising emapalumab. In some embodiments, an “undetectable” level of IFNγ is a level of 25 pg/mL or less.

In one aspect, a method of identifying a subject for treatment with emapalumab comprises the steps of (a) obtaining a sample from the subject and (b) measuring the level of IFNγ and the expression of HLA-DR on CD14 monocytes in the sample. If the expression of HLA-DR on CD14 monocytes is 8000 receptors/CD14-monocyte or more and the level of IFNγ is detectable, the subject may be determined suitable for emapalumab treatment and an initial dose of emapalumab may be administered to the subject. If the HLA-DR on CD14 monocytes expression is less than 8000 receptors/CD14-monocyte or the IFNγ level is undetectable, the subject may be determined unsuitable for emapalumab treatment. A subject deemed unsuitable for emapalumab treatment may be monitored instead of receiving emapalumab. Monitoring a subject may comprise clinical observation, further measurements of HLA-DR on CD14 monocytes and IFNγ, or conducting other tests to determine whether emapalumab administration is indicated. Alternatively, if the HLA-DR on CD14 monocytes expression is less than 8000 receptors/CD14-monocyte or the IFNγ level is undetectable, the subject may be deemed unsuitable for emapalumab treatment and the subject may receive a treatment for sepsis not comprising emapalumab. In some embodiments, an “undetectable” level of IFNγ is a level of 25 pg/mL or less.

In one aspect, a method of identifying a subject for continued treatment with emapalumab comprises repeatedly measuring the level of IFNγ and the expression of HLA-DR on CD14 monocytes in a patient receiving emapalumab. For example, levels of IFNγ and HLA-DR on CD14 monocytes may be measured every three days after the subject first receives emapalumab. If in one of these regular measurements the expression of HLA-DR on CD14 monocytes is 6000 receptors/CD14-monocyte or more and the level of IFNγ is detectable, the subject may be deemed suitable for continued treatment with emapalumab and the subject may be administered a subsequent dose of emapalumab. If in one of these regular measurements the HLA-DR on CD14 monocytes expression is less than 6000 receptors/CD14-monocyte or the IFNγ level is undetectable, the subject may be deemed unsuitable for continued emapalumab treatment and may be monitored or receive a treatment for sepsis not comprising emapalumab. In some embodiments, an “undetectable” level of IFNγ is a level of 25 pg/mL or less.

In one aspect, a method of identifying a subject for treatment with emapalumab comprises the steps of (a) obtaining a sample from the subject and (b) measuring the level of IFNγ and CXCL9 and the expression of HLA-DR on CD14 monocytes in the sample. If the expression of HLA-DR on CD14 monocytes is 8000 receptors/CD14-monocyte or more, the level of CXCL9 is more than 2200 pg/ml and the level of IFNγ is detectable, the subject may be determined suitable for emapalumab treatment and an initial dose of emapalumab may be administered to the subject. If the HLA-DR on CD14 monocytes expression is less than 8000 receptors/CD14-monocyte, the level of CXCL9 is 2200 pg/ml or less or the IFNγ level is undetectable, the subject may be determined unsuitable for emapalumab treatment. A subject deemed unsuitable for emapalumab treatment may be monitored instead of receiving emapalumab. Monitoring a subject may comprise clinical observation, further measurements of HLA-DR, CXCL9 and IFNγ, or conducting other tests to determine whether emapalumab administration is indicated. Alternatively, if the HLA-DR on CD14 monocytes expression is less than 8000 receptors/CD14-monocyte, the level of CXCL9 is 2200 pg/ml or less, or the IFNγ level is undetectable, the subject may be deemed unsuitable for emapalumab treatment and the subject may receive a treatment for sepsis not comprising emapalumab. In some embodiments, an “undetectable” level of IFNγ is a level of 25 pg/mL or less.

Other biomarkers or assays may be used as surrogate for IFNγ activity and/or may assist in classifying a subject as suffering from sepsis driven by IFNγ or sepsis driven by another mechanism, e.g. MALS, to identify subjects for treatment with emapalumab, and/or to guide treatment with emapalumab. Examples of such biomarkers include blood bioactive adrenomedullin (bioADM), calprotectin, C-reactive protein (CRP), procalcitonin, ferritin, H3.1 neutrophil extracellular traps (NETs), IFNγ-releasing assay (IGRA), IFNγ-induced protein-10 (IP-10), MR pro-adrenomedullin (MR-proADM), pancreatic stone protein (PSP), presepsin, soluble triggering receptor expressed on myeloid cells (sTREM-1) and TNF-related apoptosis inducing ligand (TRAIL) or a combination thereof. These markers may be used to generate an integrated score to guide treatment with emapalumab.

In some embodiments, additional markers may be used to identify patients likely to benefit from a method of treatment described herein. For example, physical signs allowing the calculation of the NEWS2 (new early warning 2) score and of the SAPS (simplified acute physiology score) may be collected. The NEWS2 score determines the severity of the patients' illness on a scale of 1-7, where a score of 5 or higher indicates urgent need for intervention (Welch et al., Clin Med (Lond). 2022 November; 22 (6): 509-513. The SAPS is a predictor of mortality in sepsis patients (Zhu et al., Int J Infect Dis. 2022 January: 114:135-141).

### EXAMPLES

The examples provided in this section are for illustration only and are not intended to limit the invention.

### Example 1: Development of the Interferon-Gamma Driven Sepsis as an Independent Sepsis Endotype Driving Death

This example describes the identification and characterization of IFNγ-driven sepsis (bacterial or viral) in order to enable the design a future trial for the therapeutic efficacy of emapalumab in IFNγ-driven sepsis.

### Patients and Methods

Samples coming from 14 independent cohorts from patients with sepsis over the years 2016 to 2023 were studied; 10 cohorts are coming from Greece; one cohort from Italy; and three cohorts from Germany. Patients' sampling was done according to research protocols approved by the Institutional Review Boards of participating hospitals. Approval and informed consent allowed the use of samples for broad proteomic analyses. Patients were enrolled after written informed consent provided by themselves or legal representatives. The study cohorts are summarized in Table 2.

TABLE 2
Study Cohorts

| Study | Time | | Type | Discovery | Validation | |
|---|---|---|---|---|---|---|
| acronym | period | Country | of study | set (n) | set (n) | Identifier |
| PROMPT | 2017- | Greece | All causes of | 120 | 59 | Clinicaltrials.gov |
| | 2018 | | bacterial and viral | | | NCT03295825 |
| | | | sepsis | | | |
| PROGRESS | 2017- | Greece | Sepsis by lung | 165 | 83 | EudraCT 2017- |
| | 2019 | | infections, acute | | | 002011-33 |
| | | | pyelonephritis and | | | Clinicaltrials.gov |
| | | | primary bacteremia | | | NCT03333304 |
| ImmunoSep | 2021- | Greece | Sepsis by lung | 308 | 154 | EudraCT 2020- |
| | 2023 | | infections and | | | 005768-74 |
| | | | primary bacteremia | | | Clinicaltrials.gov |
| | | | | | | NCT04990232 |
| INCLASS | 2017- | Greece | Sepsis by lung | 72 | 35 | EudraCT 2017- |
| | 2018 | | infections, | | | 001056-55 |
| | | | abdominal | | | Clinicaltrials.gov |
| | | | infections and | | | NCT03345992 |
| | | | primary bacteremia | | | |
| INTELLIGENCE-1 | 2016- | Greece | All causes of | 69 | 35 | Clinicaltrials.gov |
| | 2017 | | bacterial sepsis | | | NCT03306186 |
| ACCESS | 2021- | Greece | Sepsis by lung | 78 | 39 | EudraCT 2020- |
| | 2023 | | infections | | | 004452-15 |
| | | | | | | Clinicaltrials.gov |
| | | | | | | NCT04724044 |
| HSSG | 2016- | Greece | All causes of | 1941 | 908 | |
| | 2023 | | bacterial and viral | | | |
| | | | sepsis | | | |
| PROVIDE | 2017- | Greece | Sepsis by lung | 153 | 77 | EudraCT 2017- |
| | 2019 | | infections, | | | 002171-26 |
| | | | abdominal | | | Clinicaltrials.gov |
| | | | infections and | | | NCT03332225 |
| | | | primary bacteremia | | | |
| ESCAPE | 2020- | Greece | SARS-CoV-2 | 180 | 89 | EudraCT 2020- |
| | 2021 | | sepsis | | | 001039-29 |
| | | | | | | Clinicaltrials.gov |
| | | | | | | NCT04339712 |
| MODIFY | 2023 | Greece | Sepsis by lung | 52 | 27 | Clinicaltrials.gov |
| | | | infections, | | | NCT05909683 |
| | | | acute | | | |
| | | | pyelonephritis and | | | |
| | | | primary bacteremia | | | |
| Gemelli | 2020- | Italy | SARS-CoV-2 | 121 | 61 | |
| | 2022 | | sepsis | | | |
| SISPCT | 2018- | Germany | All causes of | 458 | 231 | Clinicaltrials.gov |
| | 2022 | | bacterial and viral | | | NCT00832039 |
| | | | sepsis | | | |
| COVID | 2020- | Germany | SARS-CoV-2 | 118 | 58 | |
| | 2022 | | sepsis | | | |
| CandiSep | 2018- | Germany | Bacterial sepsis | 36 | 18 | Clinicaltrials.gov |
| | 2022 | | | | | NCT02734550 |
| Total | | | | 3,670 | 1,833 | |
| patients | | | | | | |

All studied cohorts have common inclusion and exclusion criteria. Inclusion criteria were: a) adults (≥18 years) of either sex; b) presence of infection defined by clinical or microbiological criteria, c) sepsis defined by the Sepsis-3 definitions as any at least 2-point increase of the baseline SOFA (sequential organ failure assessment) score; and d) blood sampling the first 24 hours from sepsis onset. Sepsis onset was defined as the first timepoint the patient met the Sepsis-3 definitions. This was defined as the first 24 hours from hospital admission for patients admitted with sepsis from the emergencies or the first 24 hours from increase of the baseline SOFA score for hospitalized patients. Patients living with the human immunodeficiency virus, with neutropenia (less than 1,000 neutrophils/mm³ of whole blood), and end-stage malignancy were excluded.

The following data were captured in an electronic database: a) age and sex; b) vital signs; c) comorbidities and Charlson's comorbidity index (CCI); d) type of infection; e) APACHE (acute physiology and chronic health evaluation) II and SOFA severity scores; f) type of organ dysfunction; g) microbiology and h) laboratory data including hemoglobin, total white blood cell counts; total platelet count; international normalized ratio (INR); fibrinogen; D-dimers; C-reactive protein (CRP) and procalcitonin (PCT). Outcome (survival/death) and the exact time of survival until day 28 were also captured.

Following sampling, blood was poured into clear and pyrogen-free tubes (Vacutainer, Becton Dickinson, Cockeysville, USA) and centrifuged. Serum was kept refrigerated at −80° C. Samples were transported into a central lab for the measurement of the following proteins: a) IFNγ, interleukin (IL)-6, IP-10 (Diaclone, Besançon, France), CXCL9, interleukin (IL)-18 (ELK Biotechnology Ltd, Denver, USA) and soluble sCD163 (ELK Biotechnology) with an enzyme immunosorbent assay. The lower limits of detection were 25 pg/ml, 125 pg/ml, 312 pg/ml, 500 pg/mL and 15.6 ng/ml respectively; samples yielding concentrations of IFNγ less than 25 pg/ml, were re-measured using one high-sensitivity enzyme immunosorbent assay with lower detection limit 0.78 pg/ml (Diaclone); b) ferritin with an immunochemiluminescent assay (CLIA, DiaSorin, Saluggia, Italy) and lower detection limit 0.25 ng/ml; c) triglycerides, aspartate aminotransferases (AST) and bilirubin with an enzymatic assay on Atellica CH 930 analyzer (SIEMENS, Erlangen, Germany) and lower limit of detection 15 mg/dl, 5 IU/l and 0.15 mg/dl respectively. In some patients, blood samples collected at 72 hours from the first sampling were available; IFNγ and CXCL9 were measured in these samples.

In a subgroup of patients (Table 3), whole blood was poured in tubes covered by ethyl-diamine-tetracetic acid and incubated for 15 minutes in the dark with Anti-Human HLA-DR PE/Monocyte PerCP-Cy 5.5 (Quantibrite, BD Biosciences, Franklin Lakes, NJ, USA). The number of antibodies bound on the surface antigen HLA-DR on CD14-monocytes was counted after passage by the Navios EX (Beckman Coulter, Brea, USA) flow cytometer and was considered an expression of the absolute count of HLA-DR receptors. Patients with absolute HLA-DR receptors less than 8,000/CD14-monocyte were classified as having sepsis-induced immunoparalysis. This cut-off is derived by the CE mark provided for this IVD lab test.

TABLE 3
Study cohorts, split into discovery and validation set, for
which HLA-DR measurement on CD45/CD14-monocytes is available

| | | | | | Discovery | Validation |
|---|---|---|---|---|---|---|
| Study | Time | | Infection causing | Sepsis | population | Population |
| Acronym | period | Country | sepsis | criteria | (n) | (n) |
| ImmunoSep | 2021- | Greece | CAP, HAP, VAP, | Sepsis-3 | 305 | 154 |
| | 2023 | | BSI | | | |
| INCLASS | 2017- | Greece | CAP, VAP, IAIs and | Sepsis-3 | 33 | 15 |
| | 2018 | | BSI | | | |
| ACCESS | 2021- | Greece | CAP | Sepsis-3 | 4 | 2 |
| | 2023 | | | | | |
| Hellenic Sepsis | 2016- | Greece | CAP, HAP, VAP, | Sepsis-3 | 56 | 30 |
| Study Group | 2023 | | IAIs, BSI and viruses | | | |
| PROVIDE | 2017- | Greece | CAP, HAP, VAP, | Sepsis-3 | 142 | 71 |
| | 2019 | | IAIs, BSI | | | |
| ESCAPE | 2020- | Greece | SARS-CoV-2 | Sepsis-3 | 149 | 75 |
| | 2021 | | | | | |
| Total | | | | | 689 | 347 |
| patients | | | | | | |

AP, acute pyelonephritis;
BSI, primary bacteremia;
CAP, community-acquired pneumonia;
HAP, hospital-acquired pneumonia;
IAI, intrabdominal infection;
n, number of patients;
VAP, ventilator-associated pneumonia

### Study Endpoints

The primary study endpoint was the frequency of IDS as an independent sepsis endotype associated with 28-day mortality. The secondary study endpoint was the association of the over-time changes of the IDS endotype with 28-day outcome.

### Endotype Classification

IDS was classified as sepsis driven by IFNγ and independently associated with 28-day mortality. Since CXCL9 and/or IP-10 may also derive from tissue macrophages moving towards the process of immunoparalysis, IDS classification should involve patients with blood IFNγ with the least likelihood for the presence of immunoparalysis. IDS classifiers were the presence of all following criteria: a) the minimum blood IFNγ associated with the least likelihood for the presence of immunoparalysis; b) increases of CXCL9 and/or IP-10 above specific cut-off associated with 28-day mortality; and c) independent association with 28-day mortality in the presence of severity variables and of other sepsis endotypes.

MALS was classified as total hemophagocytosis score (HScore)≥169 and/or the co-presence of hepatobiliary dysfunction (HBD) and disseminated intravascular coagulation (DIC). HScore was calculated by adding the individual points of specific parameters (number of cytopenias, hepatosplenomegaly, core temperature, AST, fibrinogen, ferritin, triglycerides, immunosuppression). HBD was defined as total bilirubin more than 2.5 mg/dl and serum AST at least 2 times more the upper normal limit. DIC was defined as total ISTH (International Society on Thrombosis and Hemostasis) score ≥5. Patients with blood IFNγ associated with high likelihood for immunoparalysis are described as “low IFNγ (immunoparalysis). Patients who are not classified into one of the above three endotypes have blood IFNγ with minimal likelihood for the presence of immunoparalysis without CXCL9. These patients are reported as “High IFNγ without CXCL9 increase” and they are considered to represent an adaptive endotype.

### Statistical Analysis

Patients of each cohort were randomly 2:1 split into discovery and validation populations. Discovery populations of all cohorts were merged into the discovery set and validation populations of all cohorts into the validation set. All quantitative variables were expressed as mean and standard error or median and quartiles if they followed normal or non-normal distribution respectively. Comparisons between two groups were done by the Student's t-test for normal variables and by the Mann-Whitney U test for non-normal variables. Qualitative variables between two groups were compared by the Fisher's exact test.

The development of the IDS endotype was performed in the discovery cohort. According to the definition of the IDS classification all patients with IDS should have detectable IFNγ in the bloodstream. As a first step, a ROC (receiver operator characteristics curve) to identify the Youden index of IFNγ which may best separate patients with less than or ≥8000 HLA-DR receptors on CD45/CD14-monocytes. Then patients were split into those with IFNγ above or below this IFNγ concentration. As a second step, a ROC curve was plotted to identify the Youden index of IP-10 and CXCL9 which may prognosticate 28-day mortality among patients with above the IFNγ concentration defined at the first step. Then IDS was defined as patients with detectable IFNγ and CXCL9 and/or IP-10 above the estimated cut-off points.

To identify if the presence of IDS was an independent predictor for death, step-wise multivariate Cox regression analysis was done in the discovery set with 28-day mortality as the dependent variable and APACHE II, CCI and SOFA scores, organ dysfunctions, low IFNγ and the MALS and IDS endotypes as independent variables. For the purpose of the analyses, quantitative severity scores were transformed into binomial variables using as transformation cut-off the Youden index of the respective ROC curve of each score providing the best prognostication of 28-day mortality; hazard ratios (HR) and 95% confidence intervals (CIs) were calculated.

The cut-offs estimated for all variables at the discovery set were applied at the validation set. The frequencies of IDS between the discovery and the validation cohorts were compared by the Fisher's exact test. The validation of the IDS endotype was further done by one multivariate step-wise Cox regression analysis in the validation set where 28-day mortality was the dependent variable and severity scores, organ dysfunctions, low IFNγ and the MALS and IDS endotypes the independent variables. Comparisons of survival between endotypes was done by the log-rank test. Sensitivity analyses were done to define the impact of the IDS endotype in subgroups by country, type of hospitalization, type of infection, isolated pathogens and comorbidities. Odds ratios (ORs) and 95% CI were calculated according to Mentel and Haenszel.

Concentrations of CXCL9, CRP, PCT and ferritin between patients with different endotypes were compared by the Mann-Whitney U test with Bonferroni corrections for multiple testing. The relative changes of IFNγ and CXCL9 over the first 72 hours of sepsis were calculated. The ROC curves for mortality prediction were plotted and the Youden index was calculated for the estimation of the cut-off which may better predict mortality. Mortalities of patients below and above the cut-off changes of IFNγ and CXCL9 were compared by the Fisher's exact test. ORs and 95% CIs were calculated according to Mentel and Haenszel. Separate comparisons of IFNγ and of CXCL9 between baseline and day 4 were done among survivors and non-survivors using the Wilcoxon's paired test. Any p-value less than 0.050 was considered statistically significant. The p-values were corrected by Bonferroni for multiple comparisons where appropriate. Results

The total number of participants was 5,503; 3,670 patients were randomized to the discovery set; and 1,833 randomized to the validation set. Patients randomized to each set did not differ by demographics, severity scores, type of infections, comorbidities, laboratory findings, isolated pathogens and 28-day mortality (see Table 4).

TABLE 4
Main characteristics of patients of the 16 cohorts split
into the discovery set and the validation set.

| | Discovery set | | Validation set | | |
|---|---|---|---|---|---|
| | (n = 3679) | | (n = 1833) | | p-value |
| Age, years, mean (SD) | 70.1 | (15.9) | 69.6 | (15.9) | 0.252 |
| Male sex, n (%) | 1662 | (45.3) | 808 | (44.1) | 0.448 |
| APACHE II score, mean (sd) | 18.1 | (8.7) | 18.0 | (8.5) | 0.760 |
| CCI, mean (SD) | 4.11 | (2.99) | 4.08 | (2.96) | 0.738 |
| SOFA score, mean (SD) | 6.70 | (3.95) | 6.62 | (3.93) | 0.480 |
| Main infections, n (%) | | | | | |
| Community-acquired | 1099 | (29.3) | 520 | (28.4) | 0.469 |
| pneumonia | | | | | |
| Intrabdominal infection | 515 | (14.0) | 258 | (14.1) | 0.967 |
| Acute pyelonephritis | 522 | (14.2) | 261 | (14.2) | 1.00 |
| Pneumonia by SARS- | 433 | (11.8) | 216 | (11.8) | 1.00 |
| CoV-2 | | | | | |
| Hospital-acquired | 322 | (8.8) | 163 | (8.9) | 0.880 |
| pneumonia | | | | | |
| Ventilator-associated | 339 | (9.2) | 168 | (9.2) | 0.961 |
| pneumonia | | | | | |
| Primary bacteremia | 297 | (8.1) | 149 | (8.1) | 0.958 |
| Type of organ dysfunction, n (%) | | | | | |
| ARDS | 1601 | (43.6) | 804 | (44.0) | 0.818 |
| Septic shock | 1660 | (45.2) | 830 | (45.3) | 0.977 |
| Acute kidney injury | 896 | (24.4) | 429 | (23.4) | 0.412 |
| Acute coagulopathy | 760 | (21.0) | 401 | (21.9) | 0.442 |
| Main comorbidities, n (%) | | | | | |
| Type 2 diabetes mellitus | 1051 | (28.6) | 524 | (28.6) | 0.975 |
| Chronic obstructive | 522 | (14.2) | 292 | (15.9) | 0.099 |
| pulmonary disease | | | | | |
| Chronic heart failure | 759 | (20.7) | 362 | (19.7) | 0.435 |
| Chronic renal disease | 460 | (12.5) | 225 | (12.3) | 0.795 |
| Intake of corticosteroids | 337 | (9.2) | 174 | (9.5) | 0.730 |
| the last 15 days | | | | | |
| Metastatic solid tumor | 124 | (3.4) | 74 | (4.0) | 0.220 |
| malignancy | | | | | |
| Non-Hodgkin's | 52 | (1.4) | 31 | (1.7) | 0.481 |
| lymphoma | | | | | |
| Stage I/II solid tumor | 502 | (13.7) | 253 | (13.8) | 0.901 |
| malignancy | | | | | |
| Liver cirrhosis | 84 | (2.3) | 50 | (2.7) | 0.353 |
| Laboratory findings | | | | | |
| White blood cells, /mm³, | 13958.8 | (7407.8) | 13718.6 | (7235.9) | 0.266 |
| mean (SD) | | | | | |
| Procalcitonin, ng/ml, | 0.95 | (0.22-6.59) | 0.93 | (0.21-5.65) | 0.707 |
| median (Q1-Q3) | | | | | |
| CRP, mg/l, median (Q1-Q3) | 129.2 | (44.0-233.0) | 113.0 | (39.8-227.0) | 0.111 |
| Hemoglobin, g/dl, mean | 11.16 | (2.29) | 11.06 | (2.38) | 0.357 |
| (SD) | | | | | |
| Platelets, ×10³/mm³, | 225.2 | (123.5) | 224.4 | (120.8) | 0.828 |
| mean (SD) | | | | | |
| Creatinine, mg/dl, median | 1.1 | (0.8-1.8) | 1.2 | (0.8-1.9) | 0.108 |
| (Q1-Q3) | | | | | |
| International normalized | 1.19 | (1.05-1.38) | 1.18 | (1.06-1.39) | 0.633 |
| ratio, median (Q1-Q3) | | | | | |
| Fibrinogen, mg/l, mean | 539.8 | (267.1) | 520.2 | (203.8) | 0.206 |
| (SD) | | | | | |
| D-dimers, μg/l, median | 740 | (327.8-1657.5) | 788 | (341.5-1899.0) | 0.419 |
| (Q1-Q3) | | | | | |
| Aspartate | 16 | (5-37) | 16 | (5-36) | 0.817 |
| aminotransferase, U/l, | | | | | |
| median (Q1-Q3) | | | | | |
| Ferritin, ng/ml, median | 548.4 | (225.7-1229.3) | 540.1 | (226.7-1125.9) | 0.249 |
| (Q1-Q3) | | | | | |
| Most common isolated | | | | | |
| pathogens*, n (%) | | | | | |
| Escherichia coli | 365 | (9.9) | 165 | (9.0) | 0.286 |
| Klebsiella pneumoniae | 259 | (7.1) | 114 | (6.2) | 0.255 |
| Staphylococcus aureus | 218 | (5.9) | 116 | (6.3) | 0.590 |
| Pseudomonas aeruginosa | 147 | (4.0) | 73 | (4.0) | 1.00 |
| 28-day mortality, n (%) | 1187 | (32.3) | 584 | (31.9) | 0.736 |

The ROC curve identified two IFNγ concentrations with the best ability to predict the likelihood of patients to have less than 8,000 HLA-DR receptors per CD45/CD14-monocyte. The first cut-off was 3 pg/ml and the second 5 pg/ml. The cut-off of 3 pg/ml had better specificity and positive predictive value and the cut-off of 5 pg/ml had better sensitivity. Since the IDS endotype requires the concentration of IFNγ which is linked with the least risk for sepsis-induced immunoparalysis, the concentration of 3 pg/ml which provides better specificity was selected for the rest of the development of IDS.

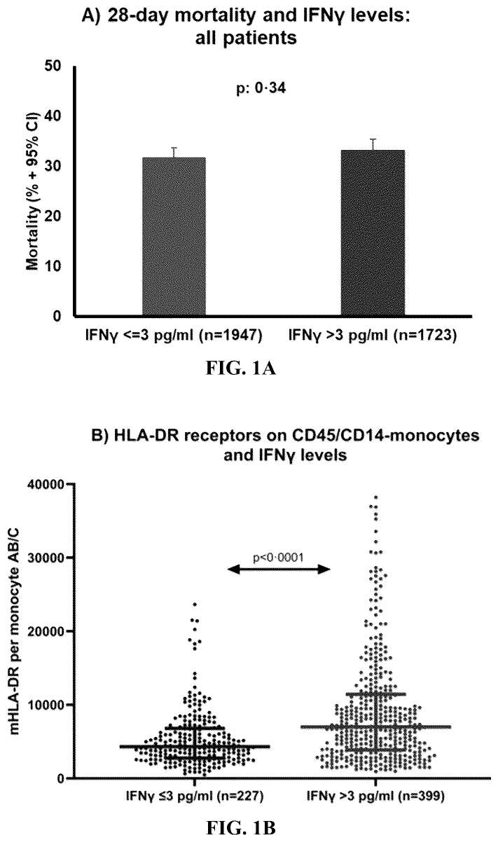

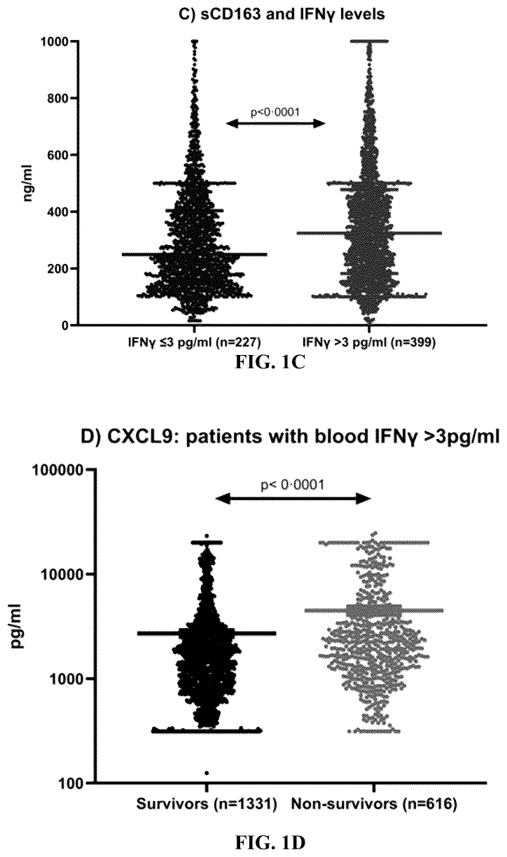

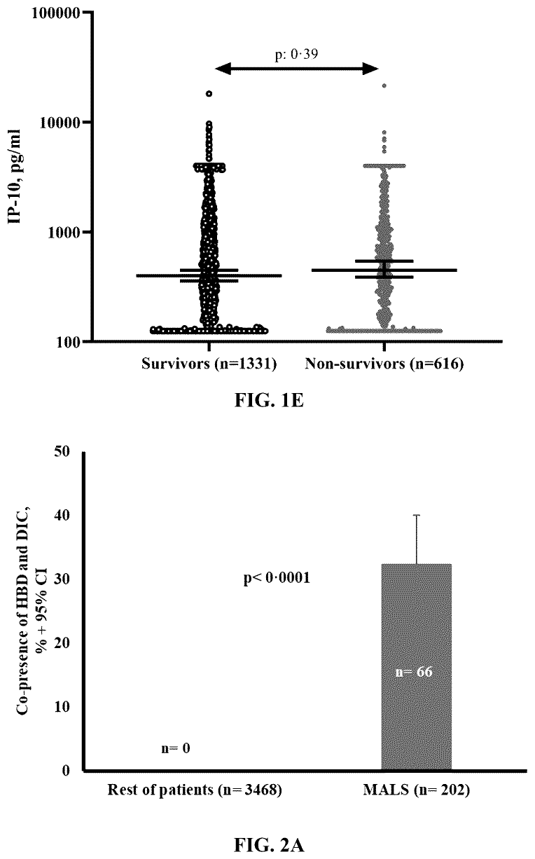

In the discovery set, significant 28-day mortality was found both for patients with IFNγ >3 pg/ml and for patients with IFNγ≤3 pg/ml. This meant that these patients represented different states of immune activation leading to death (FIG. 1A). Indeed, the absolute count of HLA-DR receptors on CD45/CD14-monocytes of patients with more than 3 pg/ml IFNγ in the blood was much higher than of patients with ≤3 pg/ml IFNγ in the blood (FIG. 1B), signifying that IFNγ≤3 pg/ml denotes the presence of immunoparalysis.

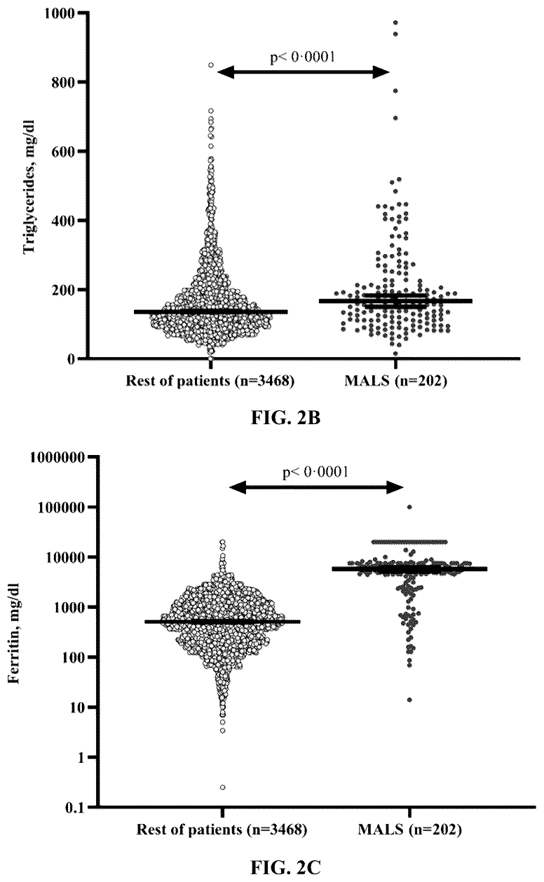

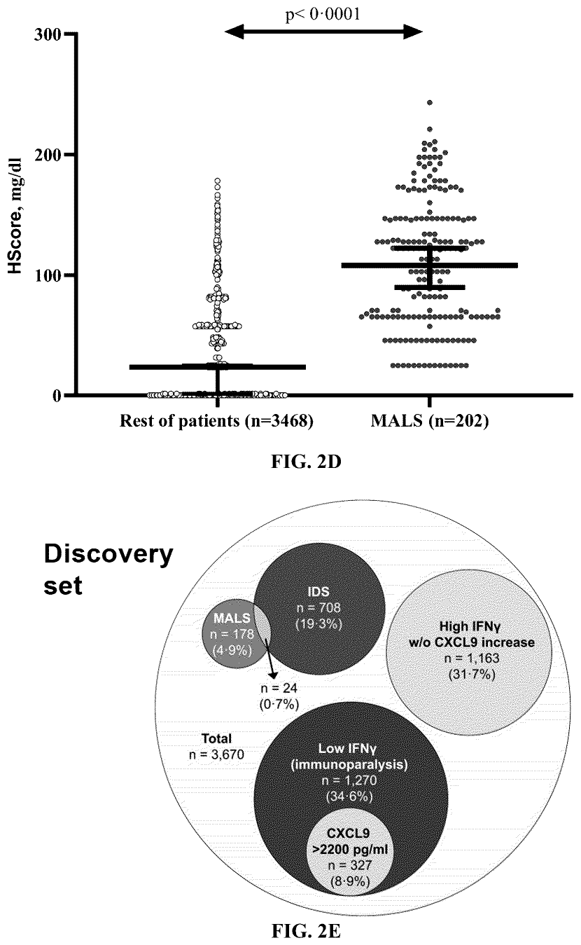

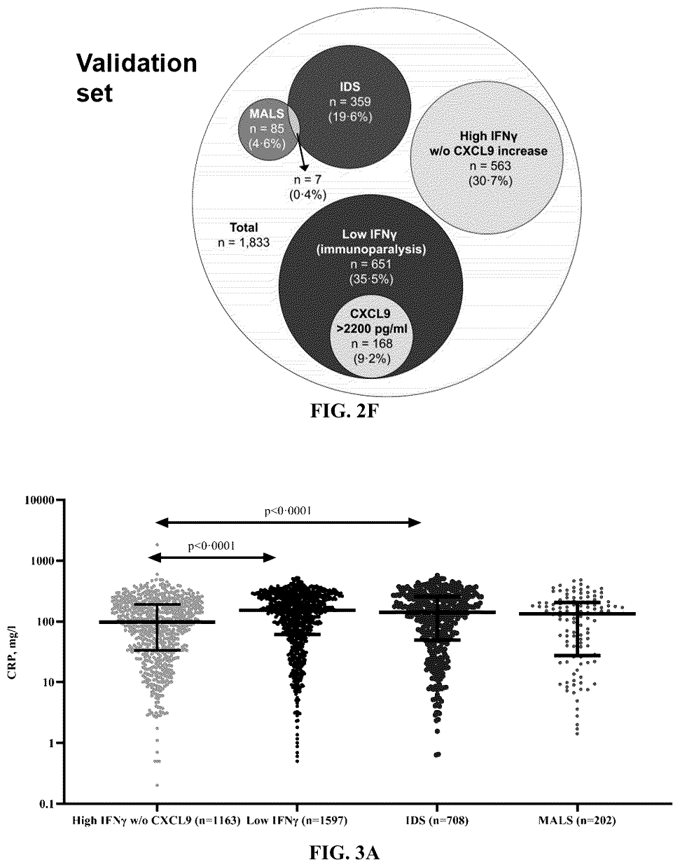

Then the analysis for the discovery of IDS was focused only on patients with IFNγ. These patients had significantly greater circulating levels of sCD163 (FIG. 1C), which is a marker of macrophage activation. CXCL9 was higher among non-survivors with IFNγ more than 3 pg/ml than among survivors (FIG. 1D). IP-10 levels did not differ among survivors and non-survivors (FIG. 1E) so that IP-10 could not be used for the development of the IDS endotype. ROC curve of CXCL9 in patients with IFNγ above 3 pg/ml identified a cut-off of 2,200 pg/ml predictable of unfavorable outcome by day 28 (area under the curve 0.63; 95% CIs 0.61 to 0.66; p<0.0001). IDS was then defined as blood IFNγ more than 3 pg/ml and blood CXCL9 above 2,200 pg/ml. Within the discovery set, patients with MALS presented with all main traits of MALS (co-presence of HBD/DIC; and increases of triglycerides, ferritin and HScore) (FIGS. 2A-2D, respectively). MALS and IDS were distinct endotypes with an overlap of only 0.7% in the discovery set (FIGS. 2E and 2F).

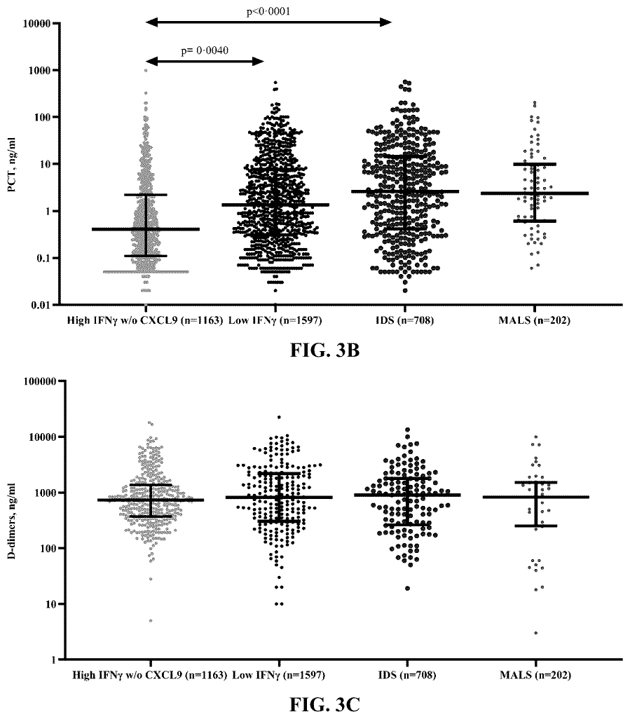

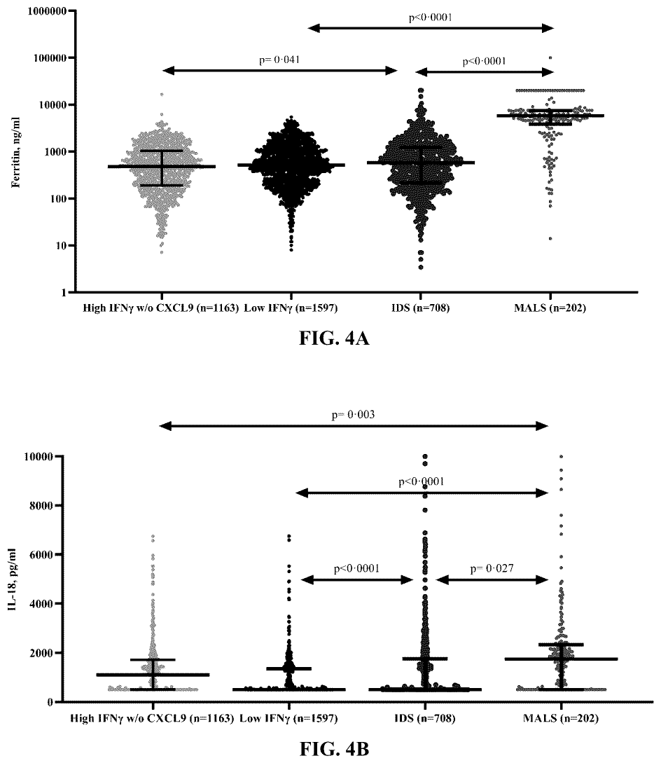

Blood CRP, PCT and D-dimers were not different between MALS and IDS (FIGS. 3A-3C). Blood ferritin and IL-18 levels were higher in MALS than IDS, and higher in IDS than the other endotypes suggesting that IDS represents a less pro-inflammatory endotype than MALS (FIGS. 4A and 4B).

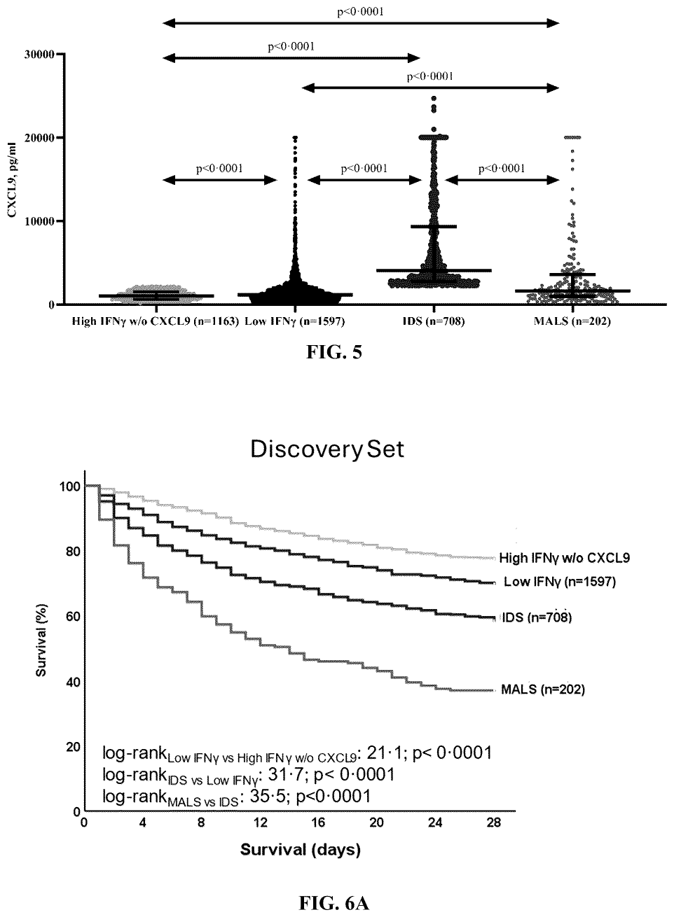

CXCL9 blood levels were significantly higher in IDS than any other endotype (FIG. 5). From the 202 patients classified with MALS at the discovery cohort, 126 patients had IFNγ≤3 pg/ml in the blood. We consider these patients as having MALS and not immunoparalysis since: a) they meet the independent classification criteria for MALS; and b) it is well described that several patients with MALS have counter decrease of HLA-DR expression on CD14-monocytes as response to high systemic tissue inflammation. These patients are classified into MALS due to the high mortality of MALS.

In both the discovery and the validation set the blood levels of IFNγ were higher in patients with IDS and in patients with high IFNγ without CXCL9 than the other endotypes (FIGS. 2E and 2F).

The frequency of IDS in the discovery set was 19.9% (732 out of 3670 patients; 95% CIs 18.7 to 21.3%). Applying the classification criteria for MALS and IDS endotypes to the validation set showed an overlap between MALS and IDS of 0.4% (FIG. 2). The frequency of IDS in the validation set was 20.0% (366 out of 1833 patients; 95% CIs 18.2 to 21.9%) (p=1.00 compared to the frequency of the discovery set). Patients with IFNγ 3 pg/ml or less were classified as a distinct endotype since these patients had a considerably different mortality. These patients probably represent patients with sepsis-induced immunoparalysis as evidenced by the low absolute count of HLA-DR receptors on CD45/CD14-monocytes (FIG. 1B). These patients could not be called sepsis-induced immunoparalysis since flow cytometry for HLA-DR on CD14-monocytes was available for a subset only and not for all patients studied.

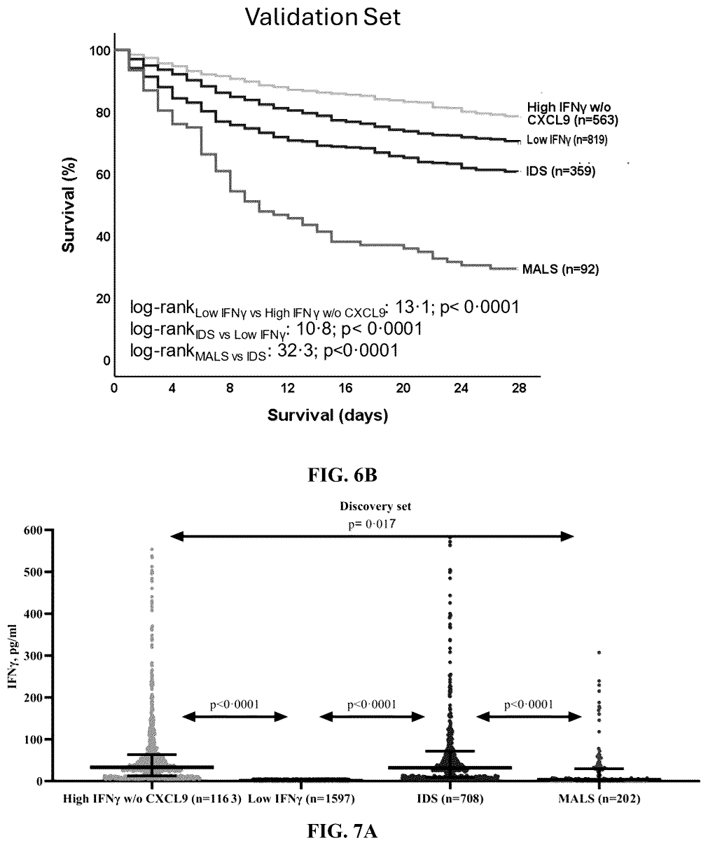

The mortality of patients with MALS was greater than for all other patients in both the discovery and the validation sets and this was followed by the mortality of patients with IDS and of patients with low IFNγ (FIGS. 6A and 6B). The mortality of patients with the IDS endotype was 43.0% (315 out of 732 patients; 95% CIs 39.5 to 46.6%) in the discovery set and 40.4% in the validation set (148 out of 366 patients; 95% CIs 35.5 to 45.5%) (p: 0.44 compared to patients of the discovery set). IDS was an independent risk factor for death under the presence of other endotypes, APACHE II, CCI and SOFA scores and of organ dysfunctions in both the discovery and the validation sets of patients (Table 5 and Table 6).

TABLE 5
Multivariate step-wise Cox regression analysis of variables associated
with 28-day mortality in the discovery set of patients

| | | | | | Univariate | | | Multivariate | | |
|---|---|---|---|---|---|---|---|---|---|---|
| | | | Non- | | analysis | | | analysis | | |
| | Survivors | | survivors | | | 95% | | | 95% | |
| Variables | n = 2483 | | n = 1187 | | HR | CIs | p-value | HR | CIs | p-value |
| Endotype, n (%) | | | | | | | | | | |
| High IFNγ without CXCL9 | 523 | (21.2) | 128 | (10.8) | 1.00 | | | 1.00 | | |
| increase (reference) | | | | | | | | | | |
| Low IFNγ | 1692 | (68.1) | 811 | (68.3) | 1.41 | 1.41-1.63 | <0.0001 | 1.17 | 1.00-1.36 | 0.038 |
| (immunoparalysis) | | | | | | | | | | |
| IFNγ-driven sepsis | 191 | (7.7) | 123 | (10.4) | 2.12 | 1.79-2.49 | <0.0001 | 1.69 | 1.43-2.00 | <0.0001 |
| Macrophage activation-like | 77 | (3.1) | 125 | (10.5) | 3.97 | 3.22-4.91 | <0.0001 | 2.05 | 1.74-2.56 | <0.0001 |
| syndrome | | | | | | | | | | |
| APACHE II ≥ 18*, n (%) | 889 | (35.8) | 800 | (67.4) | 3.00 | 2.66-3.39 | <0.0001 | 1.65 | 1.42-1.90 | <0.0001 |
| SOFA ≥ 7*, n (%) | 871 | (35.1) | 793 | (66.8) | 2.97 | 2.63-3.35 | <0.0001 | 1.51 | 1.26-1.79 | <0.0001 |
| CCI ≥ 4*, n (%) | 1252 | (50.4) | 856 | (72.1) | 2.20 | 1.91-2.50 | <0.0001 | 2.08 | 1.82-2.37 | <0.0001 |
| Presence of ARDS, n (%) | 915 | (36.9) | 685 | (57.8) | 1.97 | 1.76-2.21 | <0.0001 | 1.49 | 1.31-1.69 | <0.0001 |
| Presence of AKI, n (%) | 441 | (17.8) | 455 | (38.3) | 2.34 | 2.08-2.63 | <0.0001 | 1.47 | 1.30-1.67 | <0.0001 |
| Presence of septic shock | 925 | (37.3) | 735 | (61.9) | 2.29 | 2.04-2.59 | <0.0001 | 1.25 | 1.07-1.47 | 0.005 |
| Presence of acute | 406 | (16.4) | 363 | (30.6) | 1.96 | 1.73-2.16 | <0.0001 | 1.29 | 1.13-1.47 | <0.0001 |

coagulopathy, n (%)

*cut-off derived as the Youden index of the ROC curve to discriminate between survivors and non-survivors; Abbreviations AKI, acute kidney injury, APACHE, acute physiology and chronic health evaluation; ARDS, acute respiratory distress syndrome; CCI, Charlon's comorbidity index; CI, confidence interval; HR, hazard ratio; ROC, receiver operator characteristics; SOFA: sequential organ failure assessment.

TABLE 6
Multivariate step-wise Cox regression analysis of variables associated
with 28-day mortality in the validation set of patients

| | | | | | Univariate | | | Multivariate | | |
|---|---|---|---|---|---|---|---|---|---|---|
| | | | Non- | | analysis | | | analysis | | |
| | Survivors | | survivors | | | 95% | | | 95% | |
| Variables | n = 1249 | | n = 584 | | HR | CIs | p-value | HR | CIs | p-value |
| Endotype, n (%) | | | | | | | | | | |
| High IFNγ without CXCL9 | 257 | (20.6) | 50 | (8.6) | 1.00 | | | 1.00 | | |
| increase (reference) | | | | | | | | | | |
| Low IFNγ | 865 | (69.3) | 403 | (69.0) | 2.07 | 1.19-1.83 | <0.0001 | 1.22 | 0.98-1.52 | 0.074 |
| (immunoparalysis) | | | | | | | | | | |
| IFNγ-driven sepsis | 98 | (7.8) | 68 | (11.6) | 4.86 | 1.63-2.64 | <0.0001 | 1.69 | 1.32-2.15 | <0.0001 |
| Macrophage activation-like | 29 | (2.3) | 63 | (10.8) | 6.43 | 3.59-5.56 | <0.0001 | 2.26 | 1.65-3.11 | <0.0001 |
| syndrome | | | | | | | | | | |
| APACHE II ≥ 18*, n (%) | 460 | (36.8) | 405 | (69.3) | 3.12 | 2.62-3.72 | <0.0001 | 1.43 | 1.43-2.17 | <0.0001 |
| SOFA ≥ 7*, n (%) | 446 | (35.7) | 391 | (67.0) | 2.90 | 2.44-3.45 | <0.0001 | 1.34 | 1.05-1.71 | 0.019 |
| CCI ≥ 4*, n (%) | 632 | (50.6) | 407 | (69.7) | 1.97 | 1.65-2.35 | <0.0001 | 1.85 | 1.54-2.22 | <0.0001 |
| Presence of ARDS, n (%) | 465 | (37.2) | 341 | (58.4) | 1.99 | 1.69-2.35 | <0.0001 | 1.46 | 1.22-1.75 | <0.0001 |
| Presence of AKI, n (%) | 217 | (17.4) | 212 | (36.3) | 2.23 | 1.88-2.64 | <0.0001 | 1.46 | 1.22-1.75 | <0.0001 |
| Presence of septic shock | 458 | (36.7) | 372 | (63.7) | 2.49 | 2.10-2.95 | <0.0001 | 1.41 | 1.13-1.75 | 0.002 |
| Presence of acute | 206 | (16.5) | 195 | (33.4) | 2.17 | 1.82-2.57 | <0.0001 | 1.54 | 1.29-1.8 | <0.0001 |

coagulopathy, n (%)

*cut-off derived as the Youden index of the ROC curve to discriminate between survivors and non-survivors at the discovery set
Abbreviations AKI, acute kidney injury, APACHE, acute physiology and chronic health evaluation; ARDS, acute respiratory distress syndrome; CCI, Charlon's comorbidity index; CI, confidence interval; HR, hazard ratio; IDS, IFNγ-driven sepsis; IFN, interferon, MALS, macrophage activation-like syndrome; ROC, receiver operator characteristics; SOFA: sequential organ failure assessment

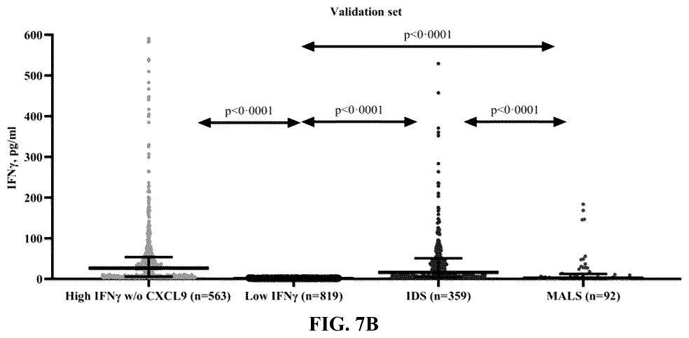

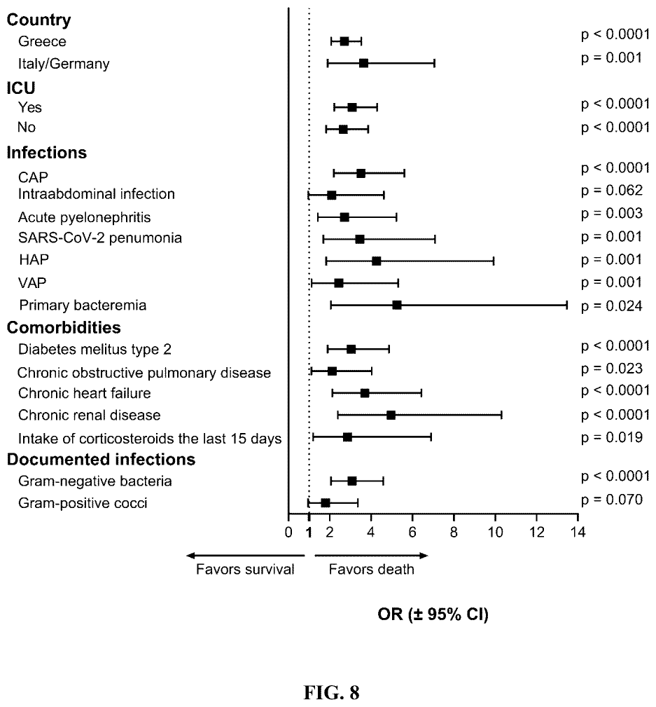

As shown in FIG. 7A for the discovery set, 327 patients with low IFNγ had CXCL9 more than 2200 pg/ml and another 1270 patients with low IFNγ had CXCL9≤2200 pg/ml; 28-day mortality was 39.4% (95% CIs 39.5 to 44.8%) and 28.5% (26.1 to 31.0%) respectively (p<0.0001). As shown in FIG. 7B for the validation set, 168 patients with low IFNγ had CXCL9 more than 2200 pg/ml and another 651 patients with low IFNγ had CXCL9≤2200 pg/ml; 28-day mortality was 43.5% (95% CIs 36.2 to 51.0%) and 27.5% (24.2 to 31.1%) respectively (p<0.0001). Sensitivity analyses revealed that the presence of IDS was an independent risk factor for death in all studied sub-groups i.e. country of origin, type of hospitalization, type of infection, comorbidities and isolated pathogens (FIG. 8).

In total, 649 patients of both cohort sets were hospitalized for pneumonia by SARS-COV-2 of which 111 patients were classified with IDS and 29 patients were classified with MALS; 51.4% and 79.3% respectively required mechanical ventilation. Among patients of the four endotypes with pneumonia by SARS-COV-2, blood ferritin was pronounced in MALS.

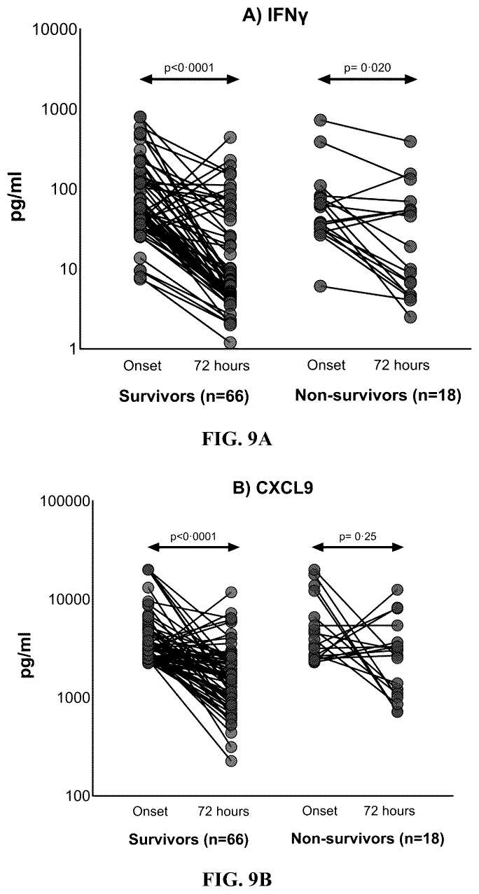

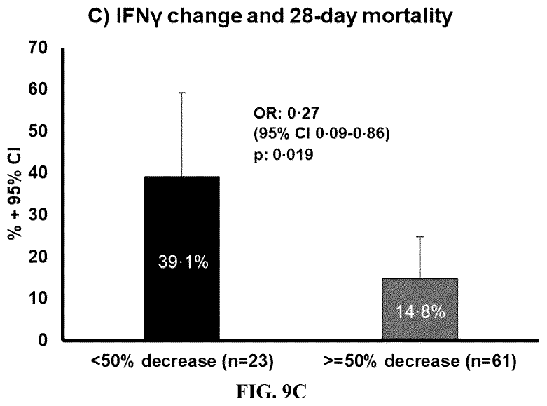

In a subset of patients with IDS, measurements of circulating IFNγ and CXCL9 were repeated after 72 hours. Although blood levels of IFNγ decrease over-time in both survivors and non-survivors, levels of CXCL9 remained stable in non-survivors (FIGS. 9A and 9B. It was found that the 28-day mortality in patients experiencing at least 50% decrease of IFNγ from baseline measurement was significantly lower than in patients with less than 40% decrease of IFNγ (FIG. 9C).

### Discussion

The present study in large cohorts of patients coming from three European countries split into one discovery set and one validation set introduces the novel IDS endotype as an independent mechanism of sepsis pathogenesis. IDS is driven by IFNγ and leads to the production of the cytotoxic CXCL9. The biomarkers defining IDS are IFNγ more than 3 pg/ml and CXCL9 more than 2200 pg/ml. IDS is found in almost 20% of patients meeting the Sepsis-3 definitions and it is an independent predictor for 28-day mortality in all patient sub-groups irrespective of the country of origin, the setting of hospitalization, the type of infection, the underlying comorbidity and the implicated pathogen. Decreases of circulating concentrations of IFNγ over the first 72 hours are linked with favorable outcomes.

The failure of randomized controlled trials (RCTs) of the past which studied the efficacy of adjuvant immunotherapies in sepsis led to the understanding that patients represent with different endotypes reflecting different mechanisms of immune activation. RCTs need to be re-designed so that patients can receive treatment tailored to their endotypes and respective clinical needs (Kyriazopoulou et al. Nature Med 2021; 27:1752-60). One recent example is the SAVE-MORE trial for COVID-19 pneumonia (Shakoory et al., Crit Care Med 2016; 44:275-81) in which patients were selected for enrolment using the biomarker suPAR (soluble urokinase plasminogen activator receptor) which surrogated the early activation of the IL-1 pathway and where patients were randomized to adjuvant treatment with either placebo or the IL-1-blocker anakinra. The relative benefit of anakinra treatment was 64%; this was the greatest recorded benefit of any drug used for COVD-19 treatment during the pandemic. The precision strategy of patient selection may have been crucial for this beneficial outcome.

In the present study three well-characterized endotypes prevailed in both the discovery and the validations set; MALS, IDS and low IFNγ, with the latter probably featuring immunoparalysis. MALS is driven by IL-1 activation (Debaugnies et al. J Clin Immunol 2021; 41:658-65).

In non-sepsis patients with secondary MAS due to solid tumor malignancies, lymphomas, autoimmune disorders and viral infections which affect the lymphoid tissues, e.g. Epstein-Barr virus and cytomegalovirus, circulating IFNγ and CXCL9 are increased (Fan, et al. Crit Care 2023; 27:347). In these patients, CXCL9 is correlated with other features of MAS like increased levels of HScore, triglycerides, ferritin, soluble IL-2 receptor (sIL-2R) and soluble receptor of tumour necrosis factor (sTNF) (Fan 2023). Our findings suggest that the markers of the IFNγ pathway in secondary MAS cannot guide the identification of the IDS endotype in sepsis. MALS and IDS are distinct because: a) the applied classification system showed 0.7% overlap in the discovery set and 0.4% in the validation set; and b) MALS is a pro-inflammatory condition with remarkable hyper-ferritinemia (Fan 2023). Although ferritin and IL-18 levels of IDS were greater than the other endotypes, they were significantly lower than in MALS, even in patients with pneumonia by SARS-COV-2, suggesting that IDS is an endotype with intermediate pro-inflammatory features between MALS and the remaining patients; and c) the cut-off of CXCL9 indicating IFNγ activation in secondary MAS is 517 pg/ml (Mizuta et al. Cytokine 2019; 119:182-7) which is much lower than the 2,200 pg/ml cut-off of IDS of sepsis. The fourth described endotype encompasses patients with blood IFNγ above 3 pg/ml and it is associated with the least risk for 28-day mortality. Most probably, this is an adaptive endotype not reflecting any specific cell activation.

Of pathomechanistic interest is the question if the deleterious effect of IDS is driven by IFNγ or by CXCL9. Tissue histology of MAS induced in mice following a challenge of the toll-like receptor 9 remained unaltered when mice had simultaneously a homogeneous deficiency for CXCL9. In a recent open-label trial, 14 patients with Still's disease refractory to corticosteroids and secondary MAS were treated with emapalumab, a monoclonal antibody which blocks the activity of IFNγ (De Benedetti et al. Ann Rheum Dis 2023; 82:857-66); MAS was improved in 50% of patients in the first 30 days; and in all patients the first 60 days. Emapalumab treatment decreased circulating CXCL9 levels suggesting that targeting IFNγ improves the syndromic entity of such patients.

Three main limitations of the present study should be acknowledged. The first limitation is that the description of IDS is based mainly on protein measurements and does not include the analysis of molecular pathways. However, transcriptomic analysis if probably unnecessary because: a) patients had increases of sCD163 indicating tissue macrophages as the effector cells of IDS (Kotsaki et al. BMJ Open 2022; 12: e067251); b) the cut-off of blood IFNγ is selected to be associated with the minimal risk of immunoparalysis, making IDS a pro-inflammatory endotype; and c) IDS was identified as an independent predictor for 28-day mortality which is a requirement for any variable of importance in sepsis biology already suggested by the Task Force of the Sepsis-3 definitions (Venet et al. Crit Care Med 2007; 35:1910-7). The second limitation is the analysis of patients with bacterial and viral sepsis together. However, analysis showed that IDS brings independent risk for death in either bacterial or viral sepsis. The third limitation is the lack of precision of the time period between the blood sampling and the onset of sepsis. This is a universal consideration in all studies which was dealt by enrolling patients with blood sampled within the first 24 hours from meeting the Sepsis-3 definitions.

The presented findings improve substantially our understanding of sepsis pathogenesis and may guide new strategies for precision treatment. This study demonstrates for the first time IDS as an entirely new endotype associated with unfavorable outcome irrespective geographic location, type of infection, type of implicated pathogen and comorbidities. IDS should be taken into consideration in all new studies of sepsis classification.

### Example 2: Emapalumab Treatment for Anticipated Clinical Benefit in Sepsis Driven by the Interferon-Gamma Endotype: The Embrace Project

This example describes a Phase IIA randomized clinical trial of the effects of emapalumab in treating interferon-gamma driven sepsis.

### Objective

As described in Example 1, an analysis of 5,503 patients with sepsis meeting the Sepsis-3 definitions and coming from Germany, Greece and Italy randomized into one discovery set and one validation set, it was found that one endotype driven by IFNγ (interferon-gamma) (IDS) is prevailing in almost 20% of patients. The presence of IDS is an independent risk for 28-day mortality irrespective the type of infection, comorbidities and organ dysfunctions. 28-day mortality is 40-43%. The EMBRACE project is aiming to unravel if treatment with emapalumab, a monoclonal which neutralizes IFNγ activity, may improve the outcome of patients with sepsis driven by the IDS endotype. EMBRACE I is the first step of this project and it aiming to identify the best dosage of emapalumab for the management of IDS.

### Study Design

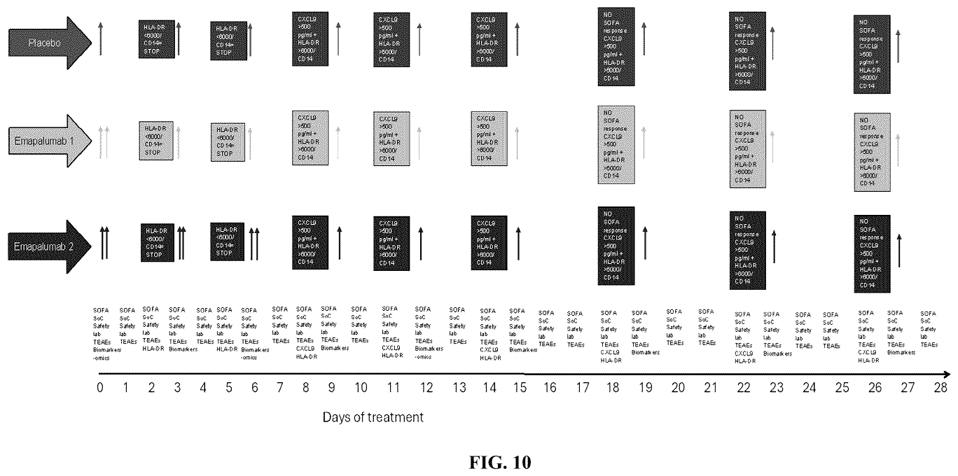

This is a double-blind, randomized, controlled, phase IIa study. Randomization will be done centrally by the electronic study website (eCRF). A summary of the study design is shown in FIG. 10. The clinical trial is published on clinicaltrials.gov under identifier NCT06694701.

### Inclusion Criteria

Provide written informed consent
Adults (≥18 years) of male or female sex
Diagnosis of community-acquired pneumonia (CAP), hospital-acquired pneumonia (HAP), ventilator-associated pneumonia (VAP), intrabdominal infection (IAI), acute pyelonephritis (AP), primary bloodstream infection (BSI) and viral respiratory infections.
Sepsis defined by the Sepsis-3 definitions. This is defined as any new infection which is accompanied by an increase of the total baseline SOFA score by at least 2 points. The total baseline SOFA score is calculated by the medical comorbidities and by the evaluation of clinical variables before the sepsis episode. In the case of patients with unknown baseline SOFA score, sepsis is defined as any new infection accompanied by total SOFA score 2 or more
Willingness to use effective contraceptive methods during the period from the start of the study drug to 6 months after the administration of the last dose of the study drug, in patients of reproductive age
Serological presence of IDS defined as detectable blood IFNγ and CXCL9 more than 2,200 pg/ml. IFNγ and CXCL9 are measured in the central study lab by an enzyme immunosorbent assay
Absence of sepsis-induced immunoparalysis (SII). This is defined as >8000 receptors of HLA-DR on CD45/CD14-monocytes measured by flow-cytometry in the central lab using the BD™ fluorescence assay

### Exclusion Criteria

Body weight more than 104 kg
Intake of any other biological during the last 30 days prior screening except for the intake of anakinra or tocilizumab for patients with active infection by SARS-COV-2
Intake of any Janus kinase inhibitors during the last 30 days prior screening except for the intake of baricitinib for patients with active infection by SARS-COV-2
Known active infection by Mycobacterium tuberculosis or other mycobacteria. These patients may be enrolled in the trial if treatment against infection by Mycobacterium tuberculosis or other mycobacteria has been initiated
Known active infection by VZV (varicella zoster virus) or by Histoplasma capsulatum or by Leishmania spp. These patients may be enrolled in the trial if treatment against infection by VZV or Histoplasma capsulatum or Leishmania spp has been initiated
Known active infection by the hepatitis B virus, by the hepatitis C virus and by cytomegalovirus
Vaccination the last 12 weeks before screening with BCG vaccine
Vaccination with any live or attenuated live vaccine (other than BCG) the last 12 weeks before screening
Known allergy or hypersensitivity reactions to emapalumab
Patients reported living with the human immunodeficiency virus (HIV)
Patients with stage IV solid or hematologic malignancy
Patients with neutropenia (less than 1,000 neutrophils/mm³)
Patients transplanted for solid organ or stem cells
Pregnancy or lactation
Participation in any other interventional trial the last 28 days prior to day 1

### Randomization

Participants are randomized in the following three groups. One stratum of randomization applies which is the presence of IAI or not. Each of the groups receives standard-of-care (SoC) treatment according to the 2021 Surviving Sepsis Campaign (SSC) guidelines. This is a phase IIa study and no power calculation can be done. It is arbitrarily selected that 25 patients will be enrolled in each group; i.e. 75 patients in total. Study Duration is 2 years.

### Placebo Group

Receiving SoC treatment and placebo treatment. Placebo treatment is administered on days 0, 3 6., 9, 12, 15, 19, 23 and 27, provided that the stopping rule does not apply.

### Emapalumab Group 1

Receiving SoC treatment and a low dose of emapalumab. Drug is administered at a dose of 6 mg/kg body weight on day 0, 3 mg/kg on day 3, 3 mg/kg on day 6, 3 mg/kg on day 9, 3 mg/kg on day 12, 3 mg/kg on day 15, 3 mg/kg on day 19, 3 mg/kg on day 23 and 3 mg/kg on day 27, provided that the stopping rule does not apply.

### Emapalumab Group 2:

Receiving SoC treatment and a high dose of emapalumab. Drug is administered at a dose of 6 mg/kg on day 0, 6 mg/kg on day 3, 6 mg/kg on day 6, 3 mg/kg on day 9, 3 mg/kg on day 12, 3 mg/kg on day 15, 3 mg/kg on day 19, 3 mg/kg on day 23 and 3 mg/kg on day 27, provided that the stopping rule does not apply.

Participants are randomized into three groups. One stratum of randomization applies which is the presence of IAI or not. Each of the groups receives standard-of-care (SoC) treatment according to the 2021 Surviving Sepsis Campaign (SSC) guidelines. The groups of randomization are the following:

Placebo administered standard-of-care (SoC) treatment and placebo drug. The drug is administered on day 0 (visit 1) and repeated dosing is provisioned for days 3, 6, 9, 12, 15, 19, 23 and 27 provided that the stopping rule does not apply.

Emapalumab group 1 administered SoC treatment and a low dose of emapalumab. The drug is administered at a dose of 6 mg/kg of body weight on day 0 (visit 1) and repeated dosing is provisioned for day 3 (3 mg/kg of body weight), day 6 (3 mg/kg of body weight), day 9 (3 mg/kg of body weight), day 12 (3 mg/kg of body weight), day 15 (3 mg/kg of body weight), day 19 (3 mg/kg of body weight), day 23 (3 mg/kg of body weight) and day 27 27 (3 mg/kg of body weight) provided that the stopping rule does not apply.

Emapalumab group 2 administered SoC treatment and a high dose of emapalumab. The drug is administered at a dose of 6 mg/kg of body weight on day 0 (visit 1) and repeated dosing is provisioned for day 3 (6 mg/kg of body weight), day 6 (6 mg/kg of body weight), day 9 (3 mg/kg of body weight), day 12 (3 mg/kg of body weight), day 15 (3 mg/kg of body weight), day 19 (3 mg/kg of body weight), day 23 (3 mg/kg of body weight) and day 27 (3 mg/kg of body weight) provided that the stopping rule does not apply.

### Stopping Rule

The applied stopping rule is as follows:
The second dose of the study drug is scheduled for day 3. If on day 2, the absolute count of HLA-DR receptors on blood CD45/CD14-monocytes is less than 6000 per cell, none of the next doses of the study drug will be administered.
The third dose of the study drug is scheduled for day 6. If on day 5, the absolute count of HLA-DR receptors on blood CD45/CD14-monocytes is less than 6000 per cell, none of the next doses of the study drug will be administered.
The fourth dose of the study drug is scheduled for day 9. If on day 8, the absolute count of HLA-DR receptors on blood CD45/CD14-monocytes is less than 6000 per cell, none of the next doses of the study drug will be administered. In case the HLA-DR on CD14 monocytes remains ≥6000 receptors per CD45/CD14-monocyte, the CXCL9 blood concentration is measured. If CXCL9 is ≤500 pg/ml none of the next doses of the study drug will be administered.
The fifth dose of the study drug is scheduled for day 12. If on day 11, the absolute count of HLA-DR receptors on blood CD45/CD14-monocytes is less than 6000 per cell, none of the next doses of the study drug will be administered. In case the HLA-DR on CD14 monocytes remains ≥6000 receptors per CD45/CD14-monocyte, the CXCL9 blood concentration is measured. If CXCL9 is ≤500 pg/ml none of the next doses of the study drug will be administered.
The sixth dose of the study drug is scheduled for day 15. If on day 14, the absolute count of HLA-DR receptors on blood CD45/CD14-monocytes is less than 6000 per cell, none of the next doses of the study drug will be administered. In case the HLA-DR on CD14 monocytes remains ≥6000 receptors per CD45/CD14-monocyte, the CXCL9 blood concentration is measured. If CXCL9 is ≤500 pg/ml none of the next doses of the study drug will be administered.
The seventh dose of the study drug is scheduled for day 19. If on day 18, the total SOFA score is decreased by at least 2 points from the baseline SOFA score before the start of the study drug, none of the next doses of the study drug will be administered. If the total SOFA score is not decreased by at least 2 points from the baseline SOFA score before the start of the study drug and the absolute count of HLA-DR receptors on blood CD45/CD14-monocytes is less than 6000 per cell, none of the next doses of the study drug will be administered. In case the HLA-DR on CD14 monocytes remains ≥6000 receptors per CD45/CD14-monocyte, the CXCL9 blood concentration is measured. If CXCL9 is ≤500 pg/ml none of the next doses of the study drug will be administered.
The eighth dose of the study drug is scheduled for day 23. If on day 22, the total SOFA score is decreased by at least 2 points from the baseline SOFA score before the start of the study drug, none of the next doses of the study drug will be administered. If the total SOFA score is not decreased by at least 2 points from the baseline SOFA score before the start of the study drug and the absolute count of HLA-DR receptors on blood CD45/CD14-monocytes is less than 6000 per cell, none of the next doses of the study drug will be administered. In case the HLA-DR on CD14 monocytes remains ≥6000 receptors per CD45/CD14-monocyte, the CXCL9 blood concentration is measured. If CXCL9 is ≤500 pg/ml none of the next doses of the study drug will be administered.
The ninth dose of the study drug is scheduled for day 27. If on day 26, the total SOFA score is decreased by at least 2 points from the baseline SOFA score before the start of the study drug, none of the next doses of the study drug will be administered. If the total SOFA score is not decreased by at least 2 points from the baseline SOFA score before the start of the study drug and the absolute count of HLA-DR receptors on blood CD45/CD14-monocytes is less than 6000 per cell, none of the next doses of the study drug will be administered. In case the HLA-DR on CD14 monocytes remains ≥6000 receptors per CD45/CD14-monocyte, the CXCL9 blood concentration is measured. If CXCL9 is ≤500 pg/ml none of the next doses of the study drug will be administered.

### Patient Follow-Up

Follow-up visits take place until day 28 and include:
Daily assessment of all serious treatment emergent adverse events (TEAEs) and of all non-serious TEAEs.
Daily calculation of the SOFA (sequential organ failure assessment) score.
Daily capture of information related to SoC treatment (e.g. microbiology, antibiotics, dose of vasopressors, ventilator settings).
Physiometrics assessment at screening.
Serum, plasma and whole blood collection at screening for measurements of exploratory diagnostic and prognostic biomarkers.
Collection of blood on days 0, 3, 6 and 12 before infusion of the study drug for transcriptomic and proteomic analyses.
Serum collection at screening and on days 3, 6, 9, 12, 15, 19, 23 and 27 before infusion of the study drug for measurements of C-reactive protein (CRP), interleukin (IL)-6, ferritin, IFNγ and CXCL9.
Collection of serum samples at 2 and 4 hours after the end of the administration of the first and second doses of study drug and 30 minutes after the end of infusion of all subsequence doses of study drug for pharmacokinetic (PK) assessment. CXCL9 will also be measured as a pharmacodynamic parameter.
Daily collection of safety lab from blood and urine. These include complete blood cell counting, renal biochemistry, liver biochemistry, glucose, electrolytes, coagulation and urinalysis.
Safety follow-up phone call visits are done 30 days (±1 day), 60 days (±3 days), 90 days (±3 days) and 120 days (±3 days) from the infusion of the first dose of the study drug.

### Endpoints:

### Primary Endpoint

The primary endpoint of the study is the decrease of SOFA score by the end-of-treatment (EOT). This is defined as either a) at least 1.4 points decrease of mean SOFA score calculated between days 1 and EOT from SOFA score of day 0; OR b) at least 2 points decrease of SOFA at EOT from day 0. Patients dying before the EOT are considered not meeting the primary endpoint. EOT is defined as the day of end of treatment of the study drug for each of the study participants. For patients requiring dosing by day 27, the decrease of the SOFA score is evaluated on day 28.

### Secondary Endpoints

Secondary endpoints supporting the primary endpoints are:
The number of doses required in each group to achieve the SOFA score response by the EOT.
28-day mortality.

The secondary endpoints of the study are the comparisons of the following between the three groups of treatment:
The rate of serious TEAEs and non-serious TEAEs.
The change of the SOFA score from day 0 until day 7.
The change of the SOFA score from day 0 until day 28.
The change of the SOFA score from day 0 until EOT.
The pharmacokinetics (i.e. drug concentrations) of emapalumab.
The need to stop the study drug due to drop of the number of HLA-DR receptors on CD14-monocytes.
The circulating concentrations of CRP, IL-6, ferritin, IFNγ and CXCL9 over the days of treatment

### Exploratory Endpoints

Exploratory endpoints of the study are:
The comparisons between the three groups of treatment for the change of the proteomic profile over treatment.
The comparisons between the three groups of treatment for the change of the transcriptomic profile over treatment.
The diagnostic performance of the point-of-care SepsisLoop for SII compared to the absolute count of HLA-DR receptor on CD45/CD14-monocytes.
The clinical data and bedside biomarker levels at screening for the potential development of a diagnostic tool classifying the study population into IDS.
The prognostic biomarker levels at screening classifying the Sepsis population in different subgroups.

### Trial Termination

Trial termination criterial are (1) completion of two years after the date of approval of Version 2.0 of the study protocol by the Greek regulatory authority or (2) one year after the end of the follow-up of the last patient; or (3) verdict by the DSMB to end the trial.

It is provisioned that the trial ends one year after the end of the follow-up of the last patient.

### Statistical Analysis

The analysis will aim to identify the best available dose of emapalumab which is associated with the best likelihood for clinical response. For this purpose the area under the curve (AUC) of the changes of the SOFA score will be correlated with the AUC of CXCL9 separately in emapalumab groups 1 and 2 between days 0 and 6. The analysis will take into consideration: a) the application of the stopping rule analyzing sub-groups for which emapalumab was stopped and sub-groups for which emapalumab was not stopped; b) the need to prolong dose administration on days 9, 12 and 15; and c) the change of CXCL9 the first 12 hours following the first and the second dose of emapalumab. The dose finding analysis will also take into consideration the outcome of patients treated with placebo and starting emapalumab on day 9 because of the high levels of CXCL9. This is done to investigate if there is time frame from IDS diagnosis and start of emapalumab which is associated with better outcomes.

1. A method of treating sepsis in a subject in need thereof, comprising administering to the subject a first dose of emapalumab, wherein the subject has a blood concentration of CXCL9 of more than 2200 pg/ml and detectable levels of IFNγ in the blood prior to the administration of the first dose of emapalumab.

2. The method of claim 1, wherein the first dose of emapalumab is a dose of about 6 mg/kg.

3. The method of claim 1, further comprising administering a second dose of emapalumab.

4. The method of claim 3, wherein the second dose is a dose of about 3 mg/kg or about 6 mg/kg.

5. (canceled)

6. The method of claim 3, wherein the second dose is administered three days after the first dose.

7. The method of claim 3, further comprising administering a third dose of emapalumab.

8. The method of claim 7, wherein the third dose is a dose of 3 mg/kg or about 6 mg/kg.

9. (canceled)

10. The method of claim 7, wherein the third dose is administered three days after the second dose.

11. The method of claim 7, further comprising administering one or more subsequent doses of emapalumab to the subject.

12. The method of claim 11, wherein the subsequent doses of emapalumab are administered to the subject every four days.

13. The method of claim 11, wherein the one or more subsequent doses of emapalumab are doses of 3 mg/kg each.

14. The method of claim 11, wherein the emapalumab is administered for a total period of 21-28 days.

15. Emapalumab for use The method of claim 1, wherein the subject has a HLA-DR expression of 8000 receptors/CD14-monocyte or more prior to the administration of the first dose of emapalumab.

16. A method of identifying a subject for treatment with emapalumab, comprising:
(a) obtaining a sample from the subject;
(b) measuring the level of IFNγ and CXCL9 in the sample; and
(c) if the level of IFNγ is detectable and the level of CXCL9 is more than 2200 pg/mL, administering at least one dose of emapalumab to the subject.

17. The method of claim 16, wherein step (c) comprises monitoring the subject or administering a therapy not comprising emapalumab if the level of IFNγ is undetectable or the level of CXCL9 is 2200 pg/ml or less.

18. The method of claim 16, comprising repeating administration of the at least one dose of emapalumab.

19. The method of claim 18, comprising repeating the administration of the at least one dose of emapalumab three times.

20. The method of claim 16, comprising administering the at least one dose of emapalumab every three or four days for 27 days.

21. The method of claim 16, further comprising measuring the level of CXCL9 at regular intervals after the administration of the at least one dose of emapalumab and, if the level of CXCL9 is more than 500 pg/ml, administering a subsequent dose of emapalumab to the subject.

22. The method of claim 21, wherein, if the level of CXCL9 is 500 pg/ml or less, the subject is monitored or treated with a therapy not comprising emapalumab.

23. The method of claim 21, wherein the level of CXCL9 is measured after the three doses of emapalumab have been administered to the subject.

24. The method of claim 16, further comprising measuring the expression of HLA-DR at regular intervals after the administration of the at least one dose of emapalumab and, if the expression of HLA-DR is 6000 receptors/CD14 monocyte or more, administering a subsequent dose of emapalumab to the subject.

25. The method of claim 24, wherein, if the expression of HLA-DR is less than 6000 receptor/monocyte, the subject is monitored or treated with a therapy not comprising emapalumab.

26. The method of claim 16, wherein the at least one dose of emapalumab is a dose of about 6 mg/kg or about 3 mg/kg.

27. (canceled)

28. (canceled)

29. The method of claim 16, wherein the subject has an expression of HLA-DR of 8000 receptors/CD14-monocyte or more prior to the administration of the at least one dose of emapalumab.

30. The method of claim 16, wherein the subject has specific values of an integrated score comprising blood C-reactive protein, procalcitonin, and/or ferritin prior to the administration of the at least one dose of emapalumab.

31-35. (canceled)


SEQUENCE LISTING (8 sequences)

SEQ ID NO: 1 - protein, 123 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGSSGWYVPHWFDPWGQGTLVTVSS

SEQ ID NO: 2 - protein, 111 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDGSNRWMFGGGTKLTVL

SEQ ID NO: 3 - protein, 5 aa, synthetic construct
SYAMS

SEQ ID NO: 4 - protein, 17 aa, synthetic construct
AISGSGGSTYYADSVKG

SEQ ID NO: 5 - protein, 14 aa, synthetic construct
DGSSGWYVPHWFDP

SEQ ID NO: 6 - protein, 13 aa, synthetic construct
TRSSGSIASNYVQ

SEQ ID NO: 7 - protein, 7 aa, synthetic construct
EDNQRPS

SEQ ID NO: 8 - protein, 10 aa, synthetic construct
QSYDGSNRWM